(19) United States
(12) Patent Application Publication
(10) Pub. No.: US 2013/0280178 A1
(43) Pub. Date: Oct. 24, 2013

(54) PROPELLANT-CONTAINING HAIR STYLING COMPOSITION

(71) Applicant: Henkel AG & Co. KGaA, Duesseldorf (DE)

(72) Inventors: Burkhard Mueller, Hamburg (DE); Uwe Bergemann, Hamburg (DE); Rolf Bayersdorfer, Hamburg (DE)

(21) Appl. No.: 13/923,933

(22) Filed: Jun. 21, 2013

Related U.S. Application Data
(63) Continuation of application No. PCT/EP2011/072193, filed on Dec. 8, 2011.

(30) Foreign Application Priority Data
Dec. 23, 2010 (DE) 10 2010 055 842.7

(51) Int. Cl. A61K 8/31; A61Q 5/06; A61K 8/04

(52) U.S. Cl. CPC A61K 8/315; A61K 8/046; A61Q 5/06
USPC 424/47

(57) ABSTRACT

An aerosol composition includes at least one setting active substance, and at least one propellant having at least one compound having 3 to 10 carbon atoms in accordance with formula (I), in which the residues R¹, R², R³, and R⁴ signify, mutually independently, a hydrogen atom, a bromine atom, a fluorine atom, or a (C₁ to C₆) alkyl group substituted with at least one fluorine atom, or two of the residues R¹, R², R³, and R⁴ form a five- or six-membered ring, with the provision that: at least one of the residues R¹, R², R³, or R⁴ denotes a hydrogen atom or a fluorine atom, and at least one of the residues R¹, R², R³, or R⁴ denotes a (C₁ to C₆) alkyl group substituted with at least one fluorine atom, or at least two of the residues R¹, R², R³, and R⁴ form a five- or six-membered ring.

### FIELD OF THE INVENTION

The present invention generally relates to temporary reshaping of keratin-containing fibers, in particular human hair. The subject matter of the invention is aerosol compositions containing at least one setting active substance and at least one special propellant, as well as the use of the compositions for the temporary reshaping of keratin-containing fibers, and a corresponding utilization method.

### BACKGROUND OF THE INVENTION

“Keratinic fibers” are understood in principle as all animal hair, e.g. wool, horsehair, angora wool, furs, feathers, and products or textiles produced therefrom. Preferably, however, the keratinic fibers are human hairs.

An attractive-looking hairstyle is generally regarded these days as an indispensable element of a well-groomed appearance. Given the currents of fashion, more and more hairstyles regarded as chic are ones that, for many types of hair, can be constructed, or maintained for a longer period of time of up to several days, only with the use of setting active substances. Hair treatment agents that serve for permanent or temporary shaping of the hair therefore play an important role. While with permanent reshaping, the chemical structure of the keratin-containing fibers is modified by reduction and oxidation, no such modification of the chemical structure takes place in the case of temporary reshaping. Corresponding agents for temporary deformation usually contain synthetic polymers and/or waxes as a setting active substance. Agents for assisting the temporary reshaping of keratin-containing fibers can be packaged, for example, as a hair spray, hair wax, hair gel, hair foam. Application by means of an aerosol delivery container, in the form of a spray or a foam, enjoys a high level of popularity.

The most important property of an agent for the temporary deformation of keratinic fibers, hereinafter also called a “styling agent,” is to impart the strongest possible hold to the treated fibers in the in the re-configured shape, i.e. in a shape imposed upon the fibers. If the keratinic fibers involved are human hairs, terms also used are a strong “hairstyle hold” or a high “degree of hold” of the styling agent. The hairstyle hold is determined substantially by the nature and quantity of the setting active substances used, although the further constituents of the styling agent, and the application form, can also have an influence.

In addition to a high degree of hold, styling agents must also meet a large number of further requirements. These can be subdivided roughly into properties on the hair; properties of the particular formulation, e.g. properties of the foam, gel, or sprayed aerosol; and properties that relate to the handling of the styling agent, the properties on the hair being of particular importance. Moisture resistance, low tack, and a balanced conditioning effect may be mentioned in particular. In addition, a styling agent should be universally usable for, if possible, all types of hair, and should be mild with respect to the hair and skin

A plurality of synthetic polymers that are utilized in styling agents have already been developed as setting active substances in order to meet the various requirements. The polymers can be subdivided into cationic, anionic, nonionic, and amphoteric setting polymers. Ideally, upon application to the hair the polymers yield a polymer film that on the one hand imparts a strong hold to the hairstyle but on the other hand is sufficiently flexible not to break under stress. If the polymer is too brittle, this results in the formation of so-called “film plaques,” i.e. residues that detach as the hair moves and give the impression that the user of the corresponding styling agent has dandruff. Similar problems result when waxes are utilized as a setting active substance in styling agents.

Application in the form of an aerosol, in particular, requires additional steps. The aerosol product must be capable of being effectively distributed onto the keratin-containing fibers, i.e. for products in the form of an aerosol spray, the composition must be capable of being uniformly applied in directed fashion as a fine spray mist. If the composition is applied in the form of an aerosol foam, that foam must be voluminous and sufficiently stable that it can be applied into the fibers, but can then correspondingly collapse on the fibers and sufficiently wet the fibers with the composition.

It is therefore desirable to make available an agent for the temporary deformation of keratin-containing fibers that is notable for a high degree of hold and can be applied effectively onto the keratin-containing fibers. It is particularly desirable for the agent to be capable of being applied onto the fibers as a fine, directed spray mist or as a rapidly collapsing foam.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An aerosol composition encompassing a preparation containing, in a cosmetically acceptable carrier, at least one setting active substance, and at least one propellant selected from at least one compound having 3 to 10 carbon atoms in accordance with formula (I)

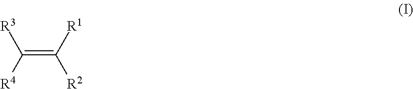

in which the residues R¹, R², R³, and R⁴ signify, mutually independently, a hydrogen atom, a bromine atom, a fluorine atom, or a (C₁ to C₆) alkyl group substituted with at least one fluorine atom, or two of the residues R¹, R², R³, and R⁴ form a five- or six-membered ring, with the provision that: at least one of the residues R¹, R², R³, or R⁴ denotes a hydrogen atom or a fluorine atom, and at least one of the residues R¹, R², R³, or R⁴ denotes a (C₁ to C₆) alkyl group substituted with at least one fluorine atom, or at least two of the residues R¹, R², R³, and R⁴ form a five- or six-membered ring.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has been found, surprisingly, that the compositions of the existing art can be optimized, in particular with regard to the aforementioned parameters, with a propellant that is selected from special fluorine-containing unsaturated carbon compounds as well as mixtures thereof.

A first subject of the invention is therefore an aerosol composition encompassing
a preparation containing, in a cosmetically acceptable carrier, at least one setting active substance, and
at least one propellant selected from at least one compound having 3 to 10 carbon atoms in accordance with formula (I)

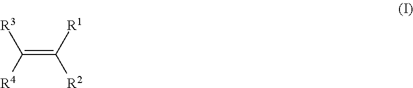

in which the residues R¹, R², R³, and R⁴ signify, mutually independently, a hydrogen atom, a bromine atom, a fluorine atom, or a (C₁ to C₆) alkyl group substituted with at least one fluorine atom,
or two of the residues R¹, R², R³, and R⁴ form a five- or six-membered ring, with the provision that:
   a. at least one of the residues R¹, R², R³, or R⁴ denotes a hydrogen atom or a fluorine atom, and
   b. at least one of the residues R¹, R², R³, or R⁴ denotes a fluorinated (C₁ to C₆) alkyl group, or at least two of the residues R¹, R², R³, and R⁴ form a five- or six-membered ring.

The preparation of the aerosol composition according to the present invention obligatorily contains at least one setting active substance. A “setting active substance” for purposes of the invention contributes, in the context of the temporary reshaping of keratin-containing fibers, to holding the shape imposed on the fibers (in the case of hair, in particular to holding a hairstyle or the hair volume). The so-called “curl retention” test is often used as a test method for the setting effect of an active substance.

For purposes of the invention, indications of quantity by weight that refer to the “preparation” are calculated based on the total weight of the preparation (i.e. without the propellant).

Preferred preparations of the aerosol composition according to the present invention contain the setting active substances in a quantity from 0.1 wt % to 25.0 wt %, particularly preferably from 1.0 wt % to 20.0 wt %, very particularly preferably from 1.0 wt % to 10.0 wt %, based in each case on the weight of said preparation.

The setting active substance of the agent according to the present invention is preferably selected from at least one setting polymer and/or from at least one wax.

In the context of an embodiment of the invention, it is preferred that the setting active substance be selected from at least one setting polymer from the group that is constituted from nonionic setting polymers, anionic setting polymers, amphoteric setting polymers, and cationic setting polymers.

In addition, the preparation according to the present invention can preferably contain at least one cationic setting polymer.

The additional cationic setting polymers comprise at least one structural unit that contains at least one permanently cationized nitrogen atom. “Permanently” cationized nitrogen atoms are to be understood as those nitrogen atoms that carry a positive charge and thereby form a quaternary ammonium compound. Quaternary ammonium compounds are usually produced by the reaction of tertiary amines with alkylating agents such as, for example, methyl chloride, benzyl chloride, dimethyl sulfate, dodecyl bromide, but also ethylene oxide. Depending on the tertiary amine used, the following groups are known in particular: alkylammonium compounds, alkenylammonium compounds, imidazolinium compounds, and pyridinium compounds.

Preparations preferred for purposes of this embodiment contain cationic setting polymers in a quantity from 0.1 wt % to 20.0 wt %, particularly preferably from 0.2 wt % to 10.0 wt %, very particularly preferably from 0.5 wt % to 5.0 wt %, based in each case on the weight of said preparation.

The cationic setting polymers can be selected, according to the present invention, from cationic quaternized cellulose derivatives.

Those cationic quaternized celluloses that carry more than one permanent cationic charge in a side chain have proven in general to be advantageous for purposes of the embodiment.

To be emphasized thereamong are, among the cationic cellulose derivatives, those that are manufactured by the reaction of hydroxyethyl cellulose with a dimethyldiallylammonium reactant (in particular dimethyldiallylammonium chloride), optionally in the presence of further reactants. Particularly suitable in turn among these cationic celluloses are those cationic celluloses having the INCI name Polyquaternium-4, which are marketed e.g. under the names Celquat® H 100, Celquat® L 200 by the National Starch company.

The following embodiments (A) to (C) are particularly preferred according to the present invention:

(A):

An aerosol composition encompassing
a preparation containing, in a cosmetically acceptable carrier, at least one cationic cellulose as a setting active substance, and
at least one propellant selected from at least one compound having 3 to 6 carbon atoms in accordance with formula (I)

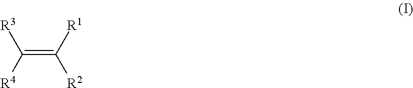

in which the residues R¹, R², R³, and R⁴ signify, mutually independently, a hydrogen atom, a bromine atom, a fluorine atom, or a (C₁ to C₆) alkyl group substituted with at least one fluorine atom,
with the provision that:
   at least one of the residues R¹, R², R³, or R⁴ denotes a hydrogen atom or a fluorine atom, and
   at least one of the residues R¹, R², R³, or R⁴ denotes a fluorinated (C₁ to C₆) alkyl group.

(B):

An aerosol composition encompassing
a preparation containing, in a cosmetically acceptable carrier, at least one cationic cellulose as a setting active substance, and
1,3,3,3-tetrafluoroprop-1-ene as a propellant.

(C):

An aerosol foam composition encompassing
a preparation containing, in a cosmetically acceptable carrier, at least one cationic cellulose as a setting active substance, and
1,3,3,3-tetrafluoroprop-1-ene as a propellant.

Also preferably suitable are those cationic setting polymers that encompass at least one structural unit of formula (M-I) and at least one structural unit of formula (M-II) and optionally at least one structural unit of formula (M-III)

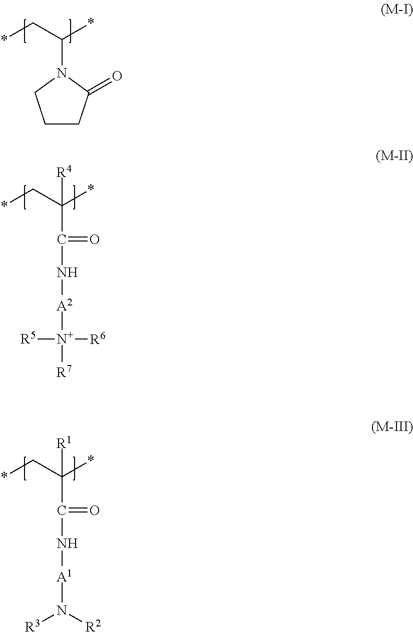

in which
R¹ and R⁴ denote, mutually independently, a hydrogen atom or a methyl group,
A¹ and A² denote, mutually independently, an ethane-1,2-diyl, propane-1,3-diyl, or butane-1,4-diyl group,
R², R³, R⁵, and R⁶ denote, mutually independently, a (C₁ to C₄) alkyl group,
R⁷ denotes a (C₈ to C₃₀) alkyl group.

All possible physiologically acceptable anions, for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to compensate for the positive charge of monomer (M-II).

Suitable compounds are commercially obtainable, for example, as
copolymers of dimethylaminoethyl methacrylate, quaternized with diethyl sulfate, with N-vinylpyrrolidone, having the INCI name Polyquaternium-11, under the designations Gafquat® 440, Gafquat® 734, Gafquat® 755 (each ISP company) and Luviquat PQ 11 PN (BASF SE),
copolymers of methacryloylaminopropyllauryldimethylammonium chloride with N-vinylpyrrolidone and dimethylaminopropyl methacrylamide, having the INCI name Polyquaternium-55, under the commercial names Styleze® W-10, Styleze® W 20 (ISP company),
copolymers of methacryloylaminopropyllauryldimethylammonium chloride with N-vinylpyrrolidone, N-vinylcaprolactam, and dimethylaminopropyl methacrylamide, having the INCI name Polyquaternium-69, under the commercial name Aquastyle® 300 (ISP company).

The following embodiments (D) to (H) are particularly preferred according to the present invention:

(D):

An aerosol composition encompassing
a preparation containing, in a cosmetically acceptable carrier, at least one cationic setting polymer that encompasses at least one structural unit of formula (M-I) and at least one structural unit of formula (M-II) and optionally at least one structural unit of formula (M-III)

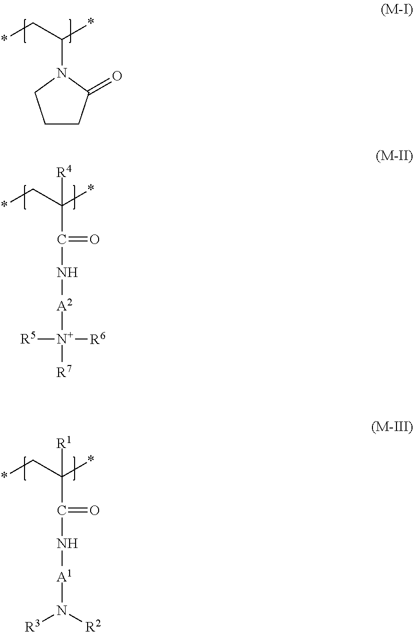

in which
R¹ and R⁴ denote, mutually independently, a hydrogen atom or a methyl group,
A¹ and A² denote, mutually independently, an ethane-1,2-diyl, propane-1,3-diyl, or butane-1,4-diyl group,
R², R³, R⁵, and R⁶ denote, mutually independently, a (C₁ to C₄) alkyl group,
R⁷ denotes a (C₈ to C₃₀) alkyl group, and
at least one propellant selected from at least one compound having 3 to 6 carbon atoms in accordance with formula (I)

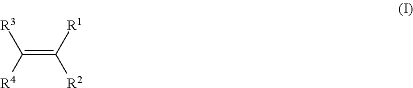

in which the residues R¹, R², R³, and R⁴ signify, mutually independently, a hydrogen atom, a bromine atom, a fluorine atom, or a (C₁ to C₆) alkyl group substituted with at least one fluorine atom,
with the provision that:
   at least one of the residues R¹, R², R³, or R⁴ denotes a hydrogen atom or a fluorine atom, and
   at least one of the residues R¹, R², R³, or R⁴ denotes a fluorinated (C₁ to C₆) alkyl group.

(E):

An aerosol composition encompassing
a preparation containing, in a cosmetically acceptable carrier, at least one cationic setting polymer that encompasses at least one structural unit of formula (M-I) and at least one structural unit of formula (M-II) and optionally at least one structural unit of formula (M-III)

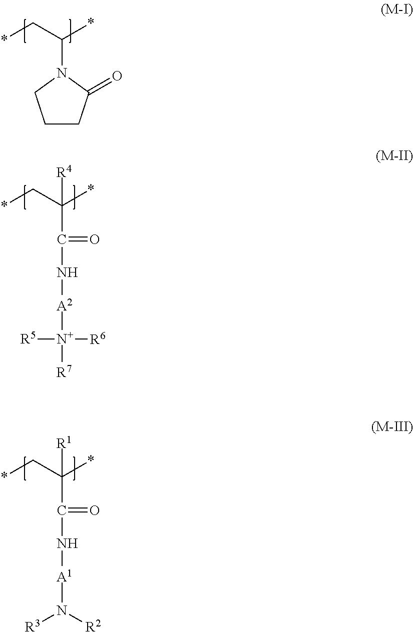

in which
R¹ and R⁴ denote, mutually independently, a hydrogen atom or a methyl group,
A¹ and A² denote, mutually independently, an ethane-1,2-diyl, propane-1,3-diyl, or butane-1,4-diyl group,
R², R³, R⁵, and R⁶ denote, mutually independently, a (C₁ to C₄) alkyl group,
R⁷ denotes a (C₈ to C₃₀) alkyl group, and
1,3,3,3-tetrafluoroprop-1-ene as a propellant.

(F):

An aerosol foam composition encompassing
a preparation containing, in a cosmetically acceptable carrier, at least one cationic setting polymer that encompasses at least one structural unit of formula (M-I) and at least one structural unit of formula (M-II) and optionally at least one structural unit of formula (M-III)

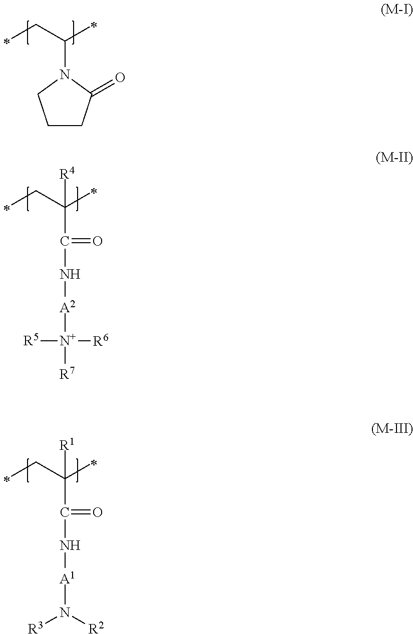

in which
R¹ and R⁴ denote, mutually independently, a hydrogen atom or a methyl group,
A¹ and A² denote, mutually independently, an ethane-1,2-diyl, propane-1,3-diyl, or butane-1,4-diyl group,
R², R³, R⁵, and R⁶ denote, mutually independently, a (C₁ to C₄) alkyl group,
R⁷ denotes a (C₈ to C₃₀) alkyl group, and
1,3,3,3-tetrafluoroprop-1-ene as a propellant.

(G):

An aerosol foam composition encompassing
a preparation containing, in a cosmetically acceptable carrier, Polyquatemium-55 as a cationic setting polymer, and
1,3,3,3-tetrafluoroprop-1-ene as a propellant.

(H):

An aerosol foam composition encompassing
a preparation containing, in a cosmetically acceptable carrier, Polyquaternium-69 as a cationic setting polymer, and
1,3,3,3-tetrafluoroprop-1-ene as a propellant.

Also serving as cationic setting polymers preferably usable for purposes of the embodiment are those cationic setting copolymers that contain at least one structural element of formula (M-IV)

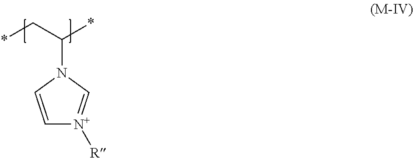

in which

R″ denotes a (C₁ to C₄) alkyl group, in particular a methyl group, and additionally comprise at least one further cationic and/or nonionic structural element.

The statements made above apply regarding compensation for the positive polymer charge.

It is furthermore preferred according to the present invention in the context of this embodiment if the preparation according to the present invention contains as a cationic setting polymer at least one copolymer (c1) that, besides at least one structural element of formula (M-IV), additionally encompasses a structural element of formula (M-I)

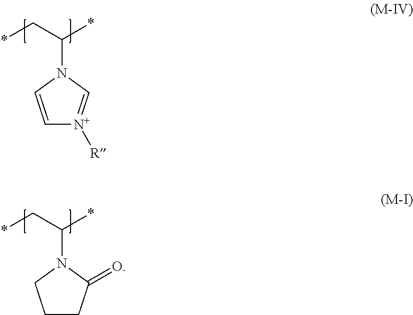

in which

R″ denotes a (C₁ to C₄) alkyl group, in particular a methyl group.

All possible physiologically acceptable anions, for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to compensate for the positive polymer charge of the copolymers (c1).

Very particularly preferred cationic setting polymers constituting copolymers (c1) contain 10 to 30 mol %, by preference 15 to 25 mol %, and in particular 20 mol % structural units according to formula (M-IV) and 70 to 90 mol %, by preference 75 to 85 mol %, and in particular 80 mol % structural units according to formula (M-I).

It is particularly preferred in this context if the copolymers (c1) contain, besides polymer units that result from incorporation of the aforesaid structural units according to formulas (M-IV) and (M-I) into the copolymer, a maximum of 5 wt %, by preference a maximum of 1 wt %, polymer units that are based on the incorporation of other monomers. By preference, the copolymers (c1) are constructed exclusively from structural units of formula (M-IV) where R″=methyl, and (M-I), and can be described by the general formula (Polyl)

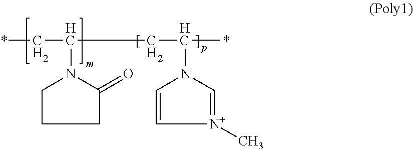

where the indices m and p each vary depending on the molar mass of the polymer and are not intended to signify that these are block copolymers. Structural units of formula (M-IV) and of formula (M-I) can instead be present in statistically distributed fashion in the molecule.

If a chloride ion is used to compensate for the positive charge of the polymer of formula (Polyl), these N-methylvinylimidazole/vinylpyrrolidone copolymers are then referred to according to INCI nomenclature as Polyquaternium-16 and are obtainable e.g. from BASF under the commercial names Luviquat® Style, Luviquat® FC 370, Luviquat® FC 550, Luviquat® FC 905, and Luviquat® HM 552.

If a methosulfate is used to compensate for the positive charge of the polymer of formula (Polyl), these N-methylvinylimidazole/vinylpyrrolidone copolymers are then referred to according to INCI nomenclature as Polyquaternium-44 and are obtainable e.g. from BASF under the commercial names Luviquat® UltraCare.

In addition to or instead of the copolymer resp. copolymers (c1), the agents according to the present invention can also contain copolymers (c2) that, proceeding from copolymer (c1), contain as additional structural units those of formula (M-V)

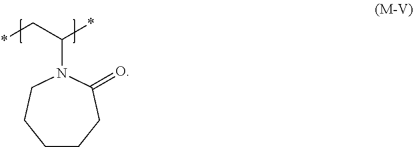

Further particularly preferred preparations according to the present invention of this embodiment are thus characterized in that they contain as a cationic setting polymer at least one copolymer (c2) that contains at least one structural unit in accordance with formula (M-IV-a) and at least a structural unit in accordance with formula (M-I) and at least a structural unit in accordance with formula (M-V)

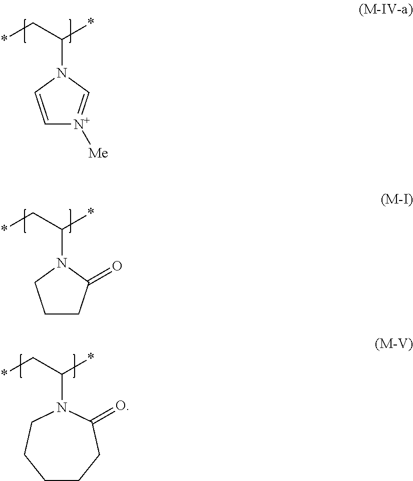

Here as well, it is particularly preferred in the context of this embodiment if the copolymers (c2) contain, besides polymer units that result from the incorporation of the aforesaid structural units according to formulas (M-IV-1), (M-I), and (M-V) into the copolymer, a maximum of 5 wt %, by preference a maximum of 1 wt %, polymer units that are based on the incorporation of other monomers. The copolymers (c2) are by preference constructed exclusively from structural units of formulas (M-IV-1), (M-I), and (M-V), and can be described by the general formula (Poly2)

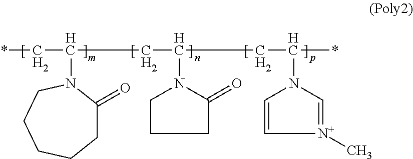

where the indices m, n and p each vary depending on the molar mass of the polymer and are not intended to signify that these are block copolymers. Structural units of the aforesaid formulas can instead be present in statistically distributed fashion in the molecule.

All possible physiologically acceptable anions, for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to compensate for the positive polymer charge of the component (c2).

If a methosulfate is used to compensate for the positive charge of the polymer of formula (Poly2), these N-methylvinylimidazole/vinylpyrrolidone/vinylcaprolactam copolymers are then referred to according to INCI nomenclature as Polyquaternium-46 and are obtainable e.g. from BASF under the commercial name Luviquat® Hold.

Very particularly preferred copolymers (c2) contain 1 to 20 mol %, by preference 5 to 15 mol %, and in particular 10 mol % structural units according to formula (M-IV-a), and 30 to 50 mol %, by preference 35 to 45 mol %, and in particular 40 mol % structural units according to formula (M-I), and 40 to 60 mol %, by preference 45 to 55 mol %, and in particular 60 mol % structural units according to formula (M-V).

In addition to or instead of the copolymer resp. copolymers (c1) and/or (c2), the agents according to the present invention can also contain, as a cationic film-forming polymer and/or cationic setting polymer, copolymers (c3) that comprise as structural units structural units of formulas (M-IV-a) and (M-I), as well as further structural units from the group of the vinylimidazole units and further structural units from the group of the acrylamide and/or methacrylamide units.

Further particularly preferred preparations according to the present invention of this embodiment are characterized in that they contain, as a cationic setting polymer, at least one copolymer (c3) that contains at least one structural unit according to formula (M-IV-a) and at least a further structural unit according to formula (M-I) and at least a further structural unit according to formula (M-VI) and at least a further structural unit according to formula (M-VII)

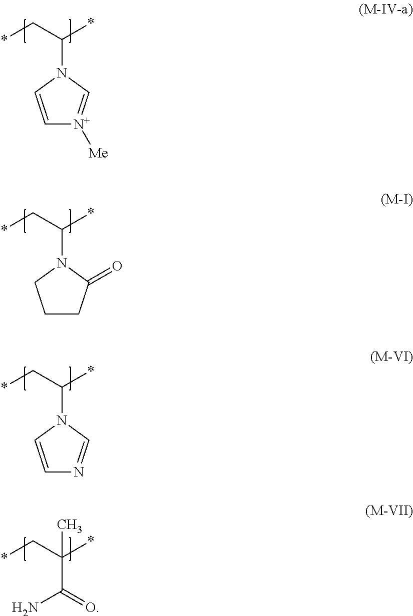

Here as well, it is particularly preferred in the context of this embodiment if the copolymers (c3) contain, besides polymer units that result from incorporation of the aforesaid structural units according to formulas (M-IV-a), (M-I), (M-VI), and (M-VII) into the copolymer, a maximum of 5 wt %, by preference a maximum of 1 wt %, polymer units that are based on the incorporation of other monomers. The copolymers (c3) are by preference constructed exclusively from structural units of formulas (M-IV-a), (M-I), (M-VI), and (M-VII) and can be described by the general formula (Poly3)

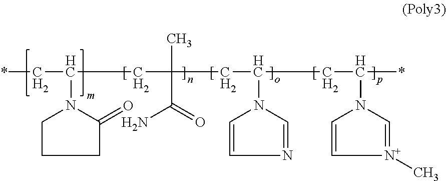

where the indices m, n, o and p each vary depending on the molar mass of the polymer and are not intended to signify that these are block copolymers. Structural units of formulas (M-IVa), (M-I), (M-VI), and (M-VII) can instead be present in statistically distributed fashion in the molecule.

All possible physiologically acceptable anions, for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to compensate for the positive polymer charge of the component (c2).

If a methosulfate is used to compensate for the positive charge of the polymer of formula (Poly3), these N-methylvinylimidazole/vinylpyrrolidone/vinylimidazole/methacrylamide copolymers are referred to according to INCI nomenclature as Polyquaternium-68 and are obtainable e.g. from BASF under the commercial name Luviquat® Supreme.

Very particularly preferred copolymers (c3) contain 1 to 12 mol %, by preference 3 to 9 mol %, and in particular 6 mol % structural units according to formula (M-IV-a), and 45 to 65 mol %, by preference 50 to 60 mol %, and in particular 55 mol % structural units according to formula (M-I), and 1 to 20 mol %, by preference 5 to 15 mol %, and in particular 10 mol % structural units according to formula (M-VI), and 20 to 40 mol %, by preference 25 to 35 mol %, and in particular 29 mol % structural units according to formula (M-VII).

Very particularly preferred copolymers (c3) contain 1 to 12 mol %, by preference 3 to 9 mol %, and in particular 6 mol % structural units according to formula (M-IV-a), and 45 to 65 mol %, by preference 50 to 60 mol %, and in particular 55 mol % structural units according to formula (M-I), and 1 to 20 mol %, by preference 5 to 15 mol %, and in particular 10 mol % structural units according to formula (M-VI), and 20 to 40 mol %, by preference 25 to 35 mol %, and in particular 29 mol % structural units according to formula (M-VII).

Among the additional setting polymers selected from the cationic polymers having at least one structural element of the above formula (M-IV), those considered preferred are:
N-vinylpyrrolidone/1-vinyl-3-methyl-1H-imidazolium chloride copolymers (such as, for example, the one having the INCI name Polyquatemium-16, under the commercial designations Luviquat® Style, Luviquat® FC 370, Luviquat® FC 550, Luviquat® FC 905, and Luviquat® HM 552 (BASF SE)),
N-vinylpyrrolidone/1-vinyl-3-methyl-1H-imidazolium methyl sulfate copolymers (such as, for example, the one having the INCI name Polyquaternium-44, under the commercial designations Luviquat® Care (BASF SE)),
N-vinylpyrrolidone/N-vinylcaprolactam/1-vinyl-3-methyl-1H-imidazolium terpolymers (such as, for example, the one having the INCI name Polyquaternium-46, under the commercial designations Luviquat® Care or Luviquat® Hold (BASF SE)),
N-vinylpyrrolidone/methacrylamide/N-vinylimidazole/1-vinyl-3-methyl-1H-imidazolium methyl sulfate copolymers (such as, for example, the one having the INCI name Polyquaternium-68, under the commercial designations Luviquat® Supreme (BASF SE)),
   as well as mixtures of said polymers.

The following embodiments (I) to (N) are particularly preferred according to the present invention:

(I):

An aerosol composition encompassing
a preparation containing, in a cosmetically acceptable carrier, at least one cationic setting polymer that encompasses, besides at least one structural element of formula (M-IV), additionally a structural element of formula (M-I)

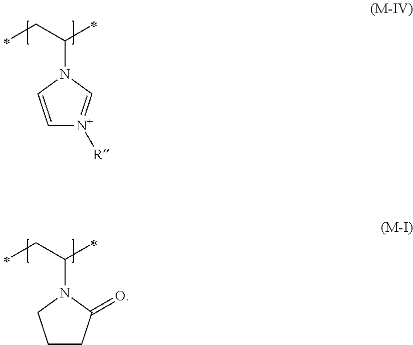

in which
   R″ denotes a (C₁ to C₄) alkyl group, in particular a methyl group, and
at least one propellant selected from at least one compound having 3 to 6 carbon atoms in accordance with formula (I)

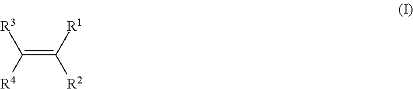

in which the residues R¹, R², R³, and R⁴ signify, mutually independently, a hydrogen atom, a bromine atom, a fluorine atom, or a (C₁ to C₆) alkyl group substituted with at least one fluorine atom,
with the provision that:
   at least one of the residues R¹, R², R³, or R⁴ denotes a hydrogen atom or a fluorine atom, and
   at least one of the residues R¹, R², R³, or R⁴ denotes a fluorinated (C₁ to C₆) alkyl group.

(J):

An aerosol composition encompassing
a preparation containing, in a cosmetically acceptable carrier, at least one cationic setting polymer that encompasses, besides at least one structural element of formula (M-IV), additionally a structural element of formula (M-I)

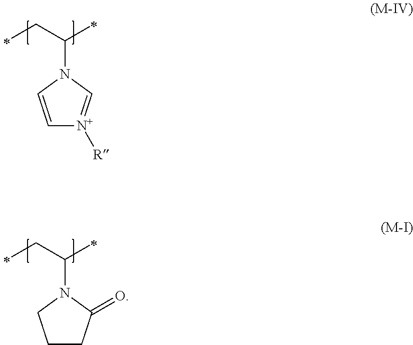

in which
   R″ denotes a (C₁ to C₄) alkyl group, in particular a methyl group, and
1,3,3,3-tetrafluoroprop-1-ene as a propellant.

(K):

An aerosol foam composition encompassing
a preparation containing, in a cosmetically acceptable carrier, at least one cationic setting polymer that encompasses, besides at least one structural element of formula (M-IV), additionally a structural element of formula (M-I)

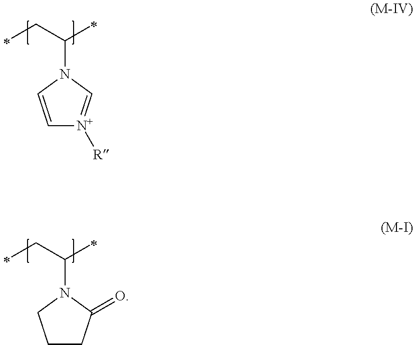

in which
   R″ denotes a (C₁ to C₄) alkyl group, in particular a methyl group, and
1,3,3,3-tetrafluoroprop-1-ene as a propellant.

(L):

An aerosol foam composition encompassing
a preparation containing, in a cosmetically acceptable carrier, Polyquaternium-16 as a cationic setting polymer, and
1,3,3,3-tetrafluoroprop-1-ene as a propellant.

(M):

An aerosol foam composition encompassing
a preparation containing, in a cosmetically acceptable carrier, Polyquaternium-44 as a cationic setting polymer, and
1,3,3,3-tetrafluoroprop-1-ene as a propellant.

(N):

An aerosol foam composition encompassing
a preparation containing, in a cosmetically acceptable carrier, Polyquaternium-68 as a cationic setting polymer, and
1,3,3,3-tetrafluoroprop-1-ene as a propellant.

The preparation according to the present invention can preferably contain at least one at least one nonionic setting polymer as an additional setting polymer. A “nonionic polymer” is understood according to the present invention as a polymer that, in a protic solvent under standard conditions, carries substantially no structural units having permanently cationic or anionic groups that must be compensated for by counterions to maintain electroneutrality. “Cationic groups” encompass, for example, quaternized ammonium groups, but not protonated amines. “Anionic groups” encompass, for example, carboxyl and sulfonic-acid groups.

In the context of a particularly preferred embodiment, the preparations contain at least one nonionic setting polymer and at least one cationic setting polymer.

The nonionic setting polymers are contained in the preparation according to the present invention of this embodiment preferably in a quantity from 0.1 wt % to 20.0 wt %, particularly preferably from 0.2 wt % to 15.0 wt %, very particularly preferably from 0.5 wt % to 10.0 wt %, based in each case on the weight of said preparation.

Those nonionic setting polymers having at least one structural element of formula (M-VIII)

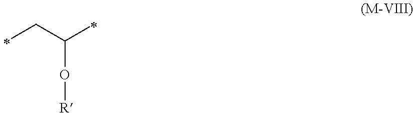

that carry, according to formula (M-VIII), a hydrogen atom, an acetyl group, or a propanoyl group, in particular an acetyl group, as R′, are particularly suitable according to the present invention.

The nonionic setting polymers are in turn preferably selected from at least one polymer of the group that is constituted from
homopolymers and nonionic copolymers of N-vinylpyrrolidone,
nonionic copolymers of isobutene.

Suitable polyvinylpyrrolidones are, for example, commercial products such as Luviskol® K 90 or Luviskol® K 85 of the BASF SE company.

Suitable polyvinyl alcohols are marketed, for example, under the commercial designations Elvanol® by Du Pont, or Vinol® 523/540 by the Air Products company.

Suitable polyvinyl acetate is marketed, for example, as an emulsion under the trade name Vinac® by the Air Products company.

Agents that contain, as a nonionic setting polymer, at least one polymer selected from the group that is constituted from
polyvinylpyrrolidone,
copolymers of N-vinylpyrrolidone and vinyl esters of carboxylic acids having 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C₁ to C₄) alkylamino-(C₂ to C₄) alkylacrylamide,
copolymers of N-vinylpyrrolidone with N,N-di(C₁ to C₄) alkylamino-(C₂ to C₄) alkylacrylamide,
   are very particularly preferred according to the present invention.

Further possible preparations according to the present invention of this embodiment having an additional nonionic setting polymer are characterized in that they contain, as a nonionic setting polymer, at least one copolymer that contains at least a further structural unit according to formula (M-I) and at least one structural unit according to formula (M-VI) and at least one structural unit according to formula (M-VII)

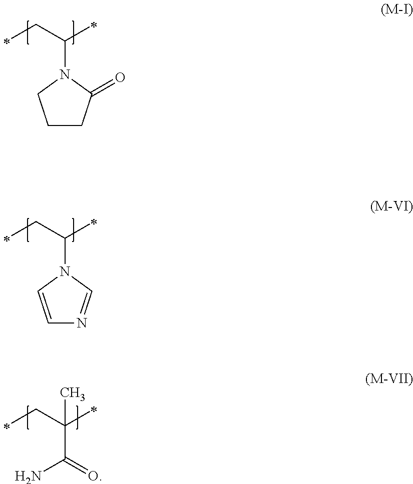

Here as well, it is particularly preferred if these copolymers contain, in addition to polymer units that result from incorporation of the aforesaid structural units according to formulas (M-IVa), (M-I), (M-VI), and (M-VII) into the copolymer, a maximum of 5 wt %, by preference a maximum of 1 wt %, polymer units that are based on the incorporation of other monomers. Copolymers (c4) are by preference constructed exclusively from structural units of formulas (M-IV-a), (M-I), (M-VI), and (M-VII) and can be described by the general formula (Poly4)

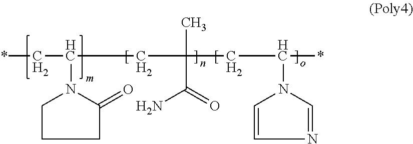

where the indices m, n, o and p each vary depending on the molecular weight of the polymer and are not intended to signify that these are block copolymers. Structural units of formulas (M-I), (M-VI), and (M-VII) can instead be present in statistically distributed fashion in the molecule.

A particularly preferred polymer is selected in this context from the polymers having the INCI name VP/Methacrylamide/Vinyl Imidazole Copolymer, which are obtainable e.g. under the trade name Luviset Clear from the BASF SE company.

The following embodiments (O) to (W) are particularly preferred according to the present invention:

(O):

An aerosol composition encompassing
a preparation containing, in a cosmetically acceptable carrier, at least one copolymer of N-vinylpyrrolidone and vinyl esters of carboxylic acids having 2 to 18 carbon atoms, in particular of N-vinylpyrrolidone and vinyl acetate, and
at least one propellant selected from at least one compound having 3 to 6 carbon atoms in accordance with formula (I)

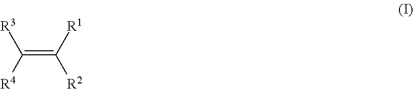

in which the residues R¹, R², R³, and R⁴ signify, mutually independently, a hydrogen atom, a bromine atom, a fluorine atom, or a (C₁ to C₆) alkyl group substituted with at least one fluorine atom,
with the provision that:
   at least one of the residues R¹, R², R³, or R⁴ denotes a hydrogen atom or a fluorine atom, and
   at least one of the residues R¹, R², R³, or R⁴ denotes a fluorinated (C₁ to C₆) alkyl group.

(P):

An aerosol composition encompassing
a preparation containing, in a cosmetically acceptable carrier, at least one copolymer of N-vinylpyrrolidone and vinyl esters of carboxylic acids having 2 to 18 carbon atoms, in particular of N-vinylpyrrolidone and vinyl acetate, and
1,3,3,3-tetrafluoroprop-1-ene as a propellant.

(Q):

An aerosol foam composition encompassing
a preparation containing, in a cosmetically acceptable carrier, at least one copolymer of N-vinylpyrrolidone and vinyl esters of carboxylic acids having 2 to 18 carbon atoms, in particular of N-vinylpyrrolidone and vinyl acetate, and
1,3,3,3-tetrafluoroprop-1-ene as a propellant.

(R):

An aerosol composition encompassing
a preparation containing, in a cosmetically acceptable carrier, polyvinylpyrrolidone, and
at least one propellant selected from at least one compound having 3 to 6 carbon atoms in accordance with formula (I)

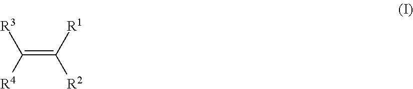

in which the residues R¹, R², R³, and R⁴ signify, mutually independently, a hydrogen atom, a bromine atom, a fluorine atom, or a (C₁ to C₆) alkyl group substituted with at least one fluorine atom,
with the provision that:
   at least one of the residues R¹, R², R³, or R⁴ denotes a hydrogen atom or a fluorine atom, and
   at least one of the residues R¹, R², R³, or R⁴ denotes a fluorinated (C₁ to C₆) alkyl group.

(S):

An aerosol composition encompassing
a preparation containing, in a cosmetically acceptable carrier, polyvinylpyrrolidone, and
1,3,3,3-tetrafluoroprop-1-ene as a propellant.

(T):

An aerosol foam composition encompassing
a preparation containing, in a cosmetically acceptable carrier, polyvinylpyrrolidone, and
1,3,3,3-tetrafluoroprop-1-ene as a propellant.

(U):

An aerosol composition encompassing
a preparation containing, in a cosmetically acceptable carrier, at least one nonionic setting polymer and at least one cationic setting polymer, and
at least one propellant selected from at least one compound having 3 to 6 carbon atoms in accordance with formula (I)

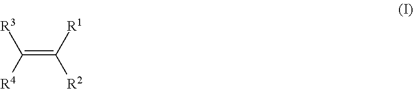

in which the residues R¹, R², R³, and R⁴ signify, mutually independently, a hydrogen atom, a bromine atom, a fluorine atom, or a (C₁ to C₆) alkyl group substituted with at least one fluorine atom,
with the provision that:
   at least one of the residues R¹, R², R³, or R⁴ denotes a hydrogen atom or a fluorine atom, and
   at least one of the residues R¹, R², R³, or R⁴ denotes a fluorinated (C₁ to C₆) alkyl group.

(V):

An aerosol composition encompassing
a preparation containing, in a cosmetically acceptable carrier, at least one nonionic setting polymer and at least one cationic setting polymer, and
1,3,3,3-tetrafluoroprop-1-ene as a propellant.

(W):

An aerosol foam composition encompassing
a preparation containing, in a cosmetically acceptable carrier, at least one nonionic setting polymer and at least one cationic setting polymer, and
1,3,3,3-tetrafluoroprop-1-ene as a propellant.

The preparations according to the present invention can also contain at least one amphoteric setting polymer as a setting polymer. The term “amphoteric polymers” encompasses both those polymers that contain in the molecule both free amino groups and free —COOH or —SO₃H groups and are capable of forming internal salts, and zwitterionic polymers, which contain quaternary ammonium groups and —COO⁻ or —SO₃⁻ groups in the molecule, and those polymers that contain —COOH or —SO₃H groups and quaternary ammonium groups.

One example of an amphoteric setting polymer usable according to the present invention is the acrylic resin obtainable under the name Amphomer®, which represents a copolymer of tert-butylaminoethyl methacrylate, N-(1,1,3,3-tetramethylbutyl)acrylamide, and two or more monomers from the group of acrylic acid, methacrylic acid, and (C₁ to C₄) alkyl esters thereof.

The latter comprise at least one negatively charged group in the molecule in addition to the cationogenic group resp. positively charged group, and are also referred to as “zwitterionic” polymers.

The amphoteric setting polymers are contained in the preparations according to the present invention preferably in quantities from 0.1 to 20 wt %, particularly preferably from 0.05 to 10 wt %, based in each case on the weight of said preparation. Quantities from 0.1 to 5 wt % are very particularly preferred.

The embodiment (X) is particularly preferred according to the present invention:

(X):

An aerosol spray composition encompassing
a preparation containing, in a cosmetically acceptable carrier, at least one copolymer of tert-butylaminoethyl methacrylate, N-(1,1,3,3-tetramethylbutyl)acrylamide, and two or more monomers from the group of acrylic acid, methacrylic acid, and (C₁ to C₄) alkyl esters thereof, and
1,3,3,3-tetrafluoroprop-1-ene as a propellant.

At least one anionic film-forming polymer can furthermore be used as setting polymers.

Anionic polymers are anionic polymers that comprise carboxylate and/or sulfonate groups. Examples of anionic monomers of which such polymers can be made up are acrylic acid, methacrylic acid, crotonic acid, maleic acid anhydride, and 2-acrylamido-2-methylpropanesulfonic acid. The acid groups can be present in this context entirely or partially as a sodium, potassium, ammonium, mono- or triethanolammonium salt.

Within this embodiment, it can be preferred to use copolymers of at least one anionic monomer and at least one nonionogenic monomer. Reference is made to the substances listed above regarding the anionic monomers. Preferred nonionogenic monomers are acrylamide, methacrylamide, acrylic acid ester, methacrylic acid ester, vinylpyrrolidone, vinyl ether, and vinyl ester.

Preferred anionic setting polymers are acrylic acid/acrylamide copolymers and in particular polyacrylamide copolymers with sulfonic acid group-containing monomers. A particularly preferred anionic setting polymer is made up of 70 to 55 mol % acrylamide and 30 to 45 mol % 2-acrylamido-2-methylpropanesulfonic acid, the sulfonic acid group being present entirely or partially as a sodium, potassium, ammonium, mono-, or triethanolammonium salt. This copolymer can also be present in crosslinked form, polyolefinically unsaturated compounds such as tetraallyoxyethane, allylsucrose, allylpentaerythritol, and methylene bisacrylamide preferably being used as crosslinking agents. One such polymer is contained in the commercial product Sepigel® 305 of the SEPPIC company. The utilization of this compound, which besides the polymer component contains a hydrocarbon mixture (C₁₃ to C₁₄ isoparaffin) and a nonionogenic emulsifier (Laureth-7), has proven particularly advantageous in the context of the teaching according to the present invention.

The sodium acryloyl dimethyl taurate copolymers marketed, under the designation Simulgel® 600, as a compound with isohexadecane and polysorbate-80 have also proven particularly effective according to the present invention.

Similarly preferred anionic setting homopolymers are uncrosslinked and crosslinked polyacrylic acids. Allyl ethers of pentaerythritol, of sucrose, and of propylene can be preferred crosslinking agents. Such compounds are obtainable commercially, for example, under the trademark Carbopol®.

Further anionic setting polymers usable in preferred fashion are selected from at least one polymer of the group that is constituted from
copolymers of vinyl acetate and crotonic acid (such as those marketed, for example, as a commercial product Aristoflex® A 60, having the INCI name VA/Crotonates Copolymer, by the CIBA company in a 60-wt % dispersion in isopropanol/water),
copolymers of ethyl acrylate and methacrylic acid (such as those marketed, for example, under the trade name Luviflex® Soft with an acid number from 84 to 105, under the INCI name Acrylates Copolymer in an approx. 20- to 30-wt % dispersion in water, by the BASF SE company),
polyurethanes having at least one carboxyl group (such as, for example, a copolymer of isophthalic acid, adipic acid, 1,6-hexanediol, neopentyl glycol, and isophorone diisocyanate, such as the one marketed under the trade name Luviset PUR, having the INCI name Polyurethane-1 or Polyurethane-34 and the commercial name Luviset Shape, by the BASF SE company).

Waxes are preferably used as a setting active substance according to the present invention. Waxes used in the context of the invention are, at 20° C., kneadable, solid to brittle-hard, coarsely to finely crystalline, transparent to opaque but not glass-like; and melt above 40° C. without decomposition. Waxes differ from similar synthetic or natural products (e.g. resins, plastic substances, metal soaps, etc.) in that from 40° C. to 90° C. they transition into the molten, low-viscosity state.

Those waxes which exhibit at 1013 mbar a melting point in the range from 50° C. to 85° C., in particular from 60° C. to 75° C., are preferred according to the present invention.

The waxes are preferably selected from vegetable, animal, and mineral waxes.

Waxes usable according to the present invention are natural waxes (vegetable waxes: cotton wax, carnauba wax, candelilla wax, esparto grass wax, guaruma wax, Japan wax, cork wax, montan wax, ouricury wax, rice seed oil wax, sugar cane wax; animal waxes: beeswax, uropygial grease, wool wax, shellac wax (see shellac), spermaceti; mineral waxes: microcrystalline waxes, ceresin, ozocerite), chemically modified waxes (hard waxes: hydrogenated jojoba waxes (see jojoba oil), montan wax, Sasol wax) and synthetic waxes (polyalkylene waxes (such as polyolefin waxes, polyethylene waxes, polypropylene waxes), polyethylene glycol waxes, amide waxes).

Waxes particularly preferred according to the present invention are beeswax (cera alba), carnauba wax, candelilla wax, montan wax, microcrystalline waxes (microcrystalline paraffins), and cetyl palmitate.

The teaching of the present invention also encompasses the combined use of multiple waxes in the preparations according to the present invention. An addition of small quantities of carnauba wax, for example, can be used to raise the melting point and drop point of another wax. A number of wax mixtures, optionally mixed with further additives, are also available commercially. Those having the designations “Spezialwachs 7686 OE” (a mixture of cetyl palmitate, beeswax, microcrystalline wax, and polyethylene, having a melting range of 73 to 75° C.; manufacturer: Kahl & Co.), Polywax® GP 200 (a mixture of stearyl alcohol and polyethylene glycol stearate, having a melting point of 47 to 51° C.; manufacturer: Croda), and “Weichceresin® FL 400” (a vaseline/vaseline oil/wax mixture having a melting point of 50 to 54° C.; manufacturer: Parafluid Mineralolgesellschaft) are examples of mixtures preferably used according to the present invention.

The preparations according to the present invention contain the waxes by preference in quantities from 1.5 to 60 wt % based on the entire preparation. Quantities from 5 to 40 wt %, in particular from 10 to 25 wt %, are particularly preferred.

Embodiments (Y) and (Z) are particularly preferred according to the present invention:

(Y):

An aerosol spray composition encompassing
a preparation containing, in a cosmetically acceptable carrier, at least one wax selected from beeswax (cera alba), carnauba wax, candelilla wax, montan wax, microcrystalline waxes (microcrystalline paraffins), and cetyl palmitate, and mixtures thereof, and
at least one propellant selected from at least one compound having 3 to 6 carbon atoms in accordance with formula (I)

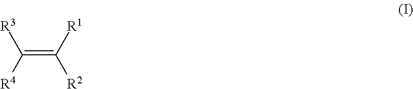

in which the residues R¹, R², R³, and R⁴ signify, mutually independently, a hydrogen atom, a bromine atom, a fluorine atom, or a (C₁ to C₆) alkyl group substituted with at least one fluorine atom,
with the provision that:
   at least one of the residues R¹, R², R³, or R⁴ denotes a hydrogen atom or a fluorine atom, and
   at least one of the residues R¹, R², R³, or R⁴ denotes a fluorinated (C₁ to C₆) alkyl group.

(Z):

An aerosol spray composition encompassing
a preparation containing, in a cosmetically acceptable carrier, at least one wax (selected from beeswax (cera alba), carnauba wax, candelilla wax, montan wax, microcrystalline waxes (microcrystalline paraffins), and cetyl palmitate, and mixtures thereof), and
1,3,3,3-tetrafluoroprop-1-ene as a propellant.

The aerosol compositions according to the present invention obligatorily contain at least one propellant of the above formula (I). It is preferred according to the present invention if the residues R¹, R², R³, and R⁴ of formula (I) denote, mutually independently, a hydrogen atom, a fluorine atom, or a fluorinated (C₁ to C₆) alkyl group substituted with at least one fluorine atom,

with the provision that:
at least one of the residues R¹, R², R³, or R⁴ denotes a hydrogen atom or a fluorine atom, and
at least one of the residues R¹, R², R³, or R⁴ denotes a fluorinated (C₁ to C₆) alkyl group.

It has further proven to be preferred according to the present invention if the propellant is selected from those compounds of formula (I) which possess 3 to 6 carbon atoms, in particular 3 or 4 carbon atoms.

It is preferred in turn that the propellant of formula (I) be selected (in particular for the embodiments (A) to (Z) preferred according to the present invention) from at least one compound of the group that is constituted from
compounds of the formula E-R¹CH═CHR² or Z—R¹CH═CHR², in which R¹ and R² represent, mutually independently, a perfluorinated C₁ to C₆ alkyl group,
CF₃CF═CHF, CF₃CH═CF₂, CHF₂CF═CF₂, CHF₂CH═CHF, CF₃CF═CH₂, CF₃CH═CHF, CH₂FCF═CF₂, CHF₂CH═CF₂, CHF₂CF═CHF, CHF₂CF═CH₂, CF₃CH═CH₂, CH₃CF═CF₂, CH₂FCH═CF₂, CH₂FCF═CHF, CHF₂CH═CHF, CF₃CF═CFCF₃, CF₃CF₂CF═CF₂, CF₃CF═CHCF₃, CF₃CF₂CF═CH₂, CF₃CH═CHF₃, CF₃CF₂CH═CH₂, CF₂═CHCF₂CF₃, CF₂═CHCF₂CF₃, CF₂═CFCHFCF₃, CF₂═CFCF₂CHF₂, CHF₂CH═CHCF₃, (CF₃)₂C═CHCF₃, CF₃CF═CHCF₂CF₃, CF₃CH═CFCF₂CF₃, (CF₃)₂CFCH═CH₂, CF₃CF₂CF₂CH═CH₂, CF₃(CF₂)₃CF═CF₂, CF₃CF₂CF═CFCF₂CF₃, (CF₃)₂C═C(CF₃)₂, (CF₃)₂CFCF═CHCF₃, CF₂═CFCF₂CH₂F, CF₂═CFCHFCHF₂, CH₂═C(CF₃)₂, CH₂CF₂CF═CF₂, CH₂FCF═CFCHF₂, CH₂FCF₂CF═CF₂, CF₂═C(CF₃)(CH₃), CH₂═C(CHF₂)(CF₃), CH₂═CHCF₂CHF₂, CF₂═C(CHF₂)(CH₃), CHF═C(CF₃)(CH₃), CH₂═C(CHF₂)₂, CF₃CF═CFCH₃, CH₃CF═CHCF₃, CF₂═CF(CF₂)₂CF₃, CHF═CF(CF₂)₂CF₃, CF₂═CH(CF₂)₂CF₃, CF₂═CF(CF₂)₂CHF₂, CHF₂CF═CFCF₂CF₃, CF₃CF═CFCF₂CHF₂, CF₃CF═CFCHFCF₃, CHF═CFCF(CF₃)₂, CF₂═CFCH(CF₃)₂, CF₃CH═C(CF₃)₂, CF₂═CHCF(CF₃)₂, CH₂═CF(CF₂)₂CF₃, CHF═CF(CF₂)₂CHF₂, CH₂═C(CF₃)C₂F₅, CF₂═CHCH(CF₃)₂, CHF═CHCF(CF₃)₂, CF₂═C(CF₃)CH₂CF₃, CH₂═CF(CF₂)₂CHF₂, CF₂═CHCF₂CH₂CF₃, CF₃CF═C(CF₃)CH₃, CH₂═CFCH(CF₃)₂, CHF═CHCH(CF₃)₂, CH₂FCH═C(CF₃)₂, CH₃CF═C(CF₃)₂, CH₂═CHCF₂CHFCF₃, CH₂═C(CF₃)CH₂CF₃, (CF₃)₂C═CHC₂F₅, CH₂═CHC(CF₃)₃, (CF₃)₂C═C(CH₃)CF₃, CH₂═CFCF₂CH(CF₃)₂, CF₃CF═C(CH₃)C₂F₅, CF₃CH═CHCH(CF₃)₂, CH₂═CH(CF₂)₃CHF₂, (CF₃)₂C═CHCF₂CH₃, CH₂═C(CF₃)CH₂C₂F₅, CH₂═CHCH₂CF₂CF₂CF₃, C₂F₅CF═CFC₂H₅, CH₂═CHCH₂CF(CF₃)₂, CF₃CF═CHCH(CF₃)(CH₃), (CF₃)₂C═CFC₂H₅, cyclo-CF₂CF₂CF₂CH═CH—, cyclo-CF₂CF₂CH═CH—, CF₃CF₂CF₂C(CH₃)═CH₂, CF₃CF₂CF₂CH═CHCH₃, cyclo-CF₂CF₂CF═CF—, cyclo-CF₂CF═CFCF₂CF₂—, cyclo-CF₂CF═CFCF₂CF₂CF₂—, CF₃CF₂CF₂CF₂CH═CH₂, CF₃CH═CHC₂F₅, C₂F₅CH═CHC₂F₅, CF₃CH═CHCF₂CF₂CF₃, CF₃CF═CFC₂F₅, CF₃CF═CFCF₂CF₂CF₂CF₃, C₂F₅CF═CFCF₂CF₂CF₃, CF₃CH═CFCF₂CF₂CF₂CF₃, CF₃CF═CHCF₂CF₂CF₂CF₃, C₂F₅CH═CFCH₂CH₂CH₃, C₂F₅CF═CHCF₂CF₂CF₃, CF₃CF₂CF₂CF═CHCH₃, C₂F₅CF═CHCH₃, (CF₃)₂C═CHCH₃, CF₃C(CH₃)═CHCF₃, CHF═CFC₂F₅, CHF₂CF═CFCF₃, (CF₃)₂C═CHF, CH₂FCF═CFCF₃, CHF═CHC₂F₅, CHF₂CH═CFCF₃, CHF═CFCHFCF₃, CF₃CH═CFCHF₂, CHF═CFCF₂CHF₂, CHF₂CF═CFCHF₂, CH₂CF═CFCF₃, CH₂FCH═CFCF₃, CH₂═CFCHFCF₃, CH₂═CFCF₂CHF₂, CF₃CH═CFCH₂F, CHF═CFCH₂CF₃, CHF═CHCHFCF₃, CHF═CHCF₂CHF₂, CHF₂CF═CHCHF₂, CHF═CFCHFCHF₂, CF₃CF═CHCH₃, CF₂═CHCF₂Br, CHF═CBrCHF₂, CHBr═CHCF₃, CF₃CBr═CFCF₃, CH₂═CBrC₂F₅, CHBr═CHC₂F₅, CH₂═CH(CF₂)₂Br, CH₂═CHCBrFCF₃, CH₃CBr═CHCF₃, CF₃CBr═CHCH₃, (CF₃)₂C═CHBr, CF₃CF═CBrC₂F₅, E-CHF₂CBr═CFC₂F₅, Z-CHF₂CBr═CFC₂F₅, CF₂═CBrCHFC₂F₅, (CF₃)₂CFCBr═CH₂, CHBr═CF(CF₂)₂CHF₂, CH₂═CBrCF₂CF₂CF₃, CF₂═C(CH₂Br)CF₃, CH₂═C(CBrF₂)CF₃, (CF₃)₂CHCH═CHBr, (CF₃)₂C═CHCH₂Br, CH₂═CHCF(CF₃)CBrF₂, CF₂═CHCF₂CH₂CBrF₂, CFBr═CHCF₃, CFBr═CFCF₃, and CH₂═CBrCF₂CF₂CF₂CF₃, in each case in the E form or the Z form.

A very particularly preferred propellant of formula (I) is represented by E-CF₃CH═CHF (E-1,3,3,3-tetrafluoroprop-1-ene).

The aerosol composition according to the present invention (in particular the preferred embodiments (A) to (Z)) contains the propellants of formula (I) preferably in a quantity from 1.0 to 60.0 wt % based on the total weight of the composition. If the agent according to the present invention is configured as an aerosol spray composition, this embodiment preferably contains 30.0 to 60.0 wt % of the propellant according to the present invention, based on the total weight of the composition. If the agent according to the present invention is configured as an aerosol foam composition, this embodiment preferably contains 1.0 to 35.0 wt %, particularly preferably 2 to 30 wt %, very particularly preferably 3 to 15 wt % propellant according to the present invention, based on the total weight of the composition.

It is also possible to use the propellant in accordance with formula (I) in combination with at least one further propellant selected from propane, propene, n-butane, isobutane, isobutene, n-pentane, pentene, isopentene, isopentene, dimethyl ether, nitrogen, air, oxygen, nitrous oxide, 1,1,1,2-tetrafluoroethane, heptafluoro-n-propane, perfluorethane, monochlorodifluoromethane, 1,1-difluoroethane, and mixtures of these propellants.

Aerosol compositions preferred according to the present invention additionally contain, besides the propellant in accordance with formula (I) defined above, dimethyl ether as a further propellant.

If an additional propellant different from the propellants of formula (I) is used, it is in turn preferred (in particular for the preferred embodiments (A) to (Z)) if the weight ratio of the propellant in accordance with formula (I) to the remaining additional propellants is greater than or equal to 1, in particular greater than or equal to 2.

The aerosol composition according to the present invention is packaged in an aerosol delivery container constituting a pressure container. A “pressure container” according to the present invention is a container which has in the interior a higher gas pressure than outside the container, and from which a gas flow can be withdrawn through a valve. Pressure containers with which a product (e.g. a liquid composition) can be delivered through a valve as a result of the internal gas pressure of the container are referred to by definition as “aerosol delivery containers.” A “non-aerosol delivery container” is defined, conversely to the “aerosol” definition, as a vessel under standard pressure with which a product is distributed by means of mechanical action by way of a pump system or squeeze system.

The aerosol compositions according to the present invention can be manufactured in usual fashion. As a rule all the constituents of the preparation of the aerosol composition according to the present invention are introduced into a suitable pressure-tight container. The latter is then sealed with a valve. Lastly, the desired quantity of the special propellant is introduced using conventional techniques.

Vessels made of metal (aluminum, tinplate, tin), shielded resp. shatterproof plastic, or glass externally coated with plastic, are suitable as pressure-tight containers; pressure-tightness and breakage resistance, corrosion resistance, easy fillability, as well as aesthetic considerations, handling, imprintability, etc., play a role in the selection thereof. Special internal protective lacquers ensure corrosion resistance with respect to the agent packaged in the pressure container Particularly preferably, the valves that are used comprise an internally lacquered valve plate, the lacquer coating and valve material being compatible with one another. If aluminum valves are used, their valve plates can then be coated internally with, for example, Microflex lacquer. If tinplate valves are used according to the present invention, their valve plates can then be internally coated with, for example, polyethylene terephthalate (PET).

The compositions according to the present invention can also be packaged in a multi-chamber dispenser. The multi-chamber dispenser can also be used in such a way that one chamber is filled with the compressed propellant and the other chamber with the remaining constituents of the aerosol composition according to the present invention. A multi-chamber dispenser of this kind is, for example, a so-called “bag-in-can” package.

If the aerosol compositions according to the present invention are configured as aerosol spray compositions, the aerosol delivery containers preferably comprise a stem valve having a stem orifice from 1×0.2 mm to 1×0.7 mm.

The valves in turn preferably have a VPH orifice from 0.00 mm to 0.60 mm.

The valves in turn preferably have an RTP diameter from 0.30 to 1.60 mm.

The following valves are preferably suitable according to the present invention:
Coster model KPM (stem orifice from 1×0.27 mm to 1×0.60 mm)
Coster model KRA (stem orifice from 1×0.27 mm to 1×0.60 mm, in combination with VPH orifices from 0.00 mm to 0.60 mm and RTP diameters from 0.60 to 1.60 mm)
Coster model RKRA (stem orifice from 1×0.27 mm to 1×0.60 mm, in combination with VPH orifices from 0.00 mm to 0.60 mm and RTP diameters from 0.30 to 1.60 mm)
Coster model T (stem orifice from 1×0.30 mm to 1×0.70 mm)
Coster model TRA (stem orifices from 1×0.30 mm to 1×0.70 mm, in combination with VPH orifices from 0.00 mm to 0.60 mm and RTP diameters from 0.30 to 1.60 mm)
Coster model RTRA (stem orifices from 1×0.30 mm to 1×0.70 mm, in combination with VPH orifices from 0.00 mm to 0.60 mm and RTP diameters from 0.30 to 1.60 mm)
Coster model KEN (stem orifice from 1×0.27 to 1×0.60 mm)
Coster model RKEN (stem orifice from 1×0.27 mm to 1×0.60 mm, in combination with VPH orifices from 0.00 mm to 0.60 mm and RTP diameters from 0.30 to 1.60 mm)
Precision Standard (with stem orifices 0.010″ to 0.024″ in combination with bottom openings 0.018″ to 0.040″)
Precision tilt valve (with stem orifices 0.010″ to 0.024″ in combination with bottom openings from 0.018″ to 0.040″)
SeaquistPerfect Ariane VX or Ariane XT (stem orifice from 1×0.25 mm to 1×0.60 mm, in combination with VPH orifices from 0.33 mm to 0.80 mm and housing bores 0.40 to 1.60 mm)

Spray heads having swirl nozzles, particularly preferably so-called “mechanical break-up” (MBU) swirl nozzles, are preferably suitable according to the present invention for embodying an aerosol spray according to the present invention, for example the V06.212 nozzle of the Coster company, the MBU Soft 0.020″ swirl nozzle of the Precision company, or the WAX spray head with DU 25 and 27.

The spray rate in the embodiment as an aerosol spray is preferably 6.5 to 10.0 g/10 s.

The preparations according to the present invention contain the ingredients resp. active substances in a cosmetically acceptable carrier.

Preferred cosmetically acceptable carriers are aqueous, alcoholic, or aqueous alcoholic media having by preference at least 5 wt % water, based on the entire preparation. The alcohols contained can be, in particular, the lower alcohols having 1 to 4 carbon atoms usually used for cosmetic purposes, for example ethanol and isopropanol.

The water content of the preparations according to the present invention is preferably 5 to 97 wt %; in the embodiment as an aerosol spray, preferably 5 to 30 wt %; in the embodiment as an aerosol foam, preferably 30 to 95 wt %.

In the context of a preferred embodiment of the preparation according to the present invention, the preparation therefore additionally contains at least one alcohol that has 2 to 6 carbon atoms and 1 to 3 hydroxyl groups. This additional alcohol is in turn preferably selected from at least one compound of the group that is constituted from ethanol, ethylene glycol, isopropanol, 1,2-propylene glycol, 1,3-propylene glycol, glycerol, n-butanol, 1,3-butylene glycol. A very particularly preferred alcohol is ethanol.

The additional alcohol having 2 to 6 carbon atoms and 1 to 3 hydroxyl groups is contained in the preparation according to the present invention preferably in a quantity from 40 wt % to 65 wt %, in particular from 40 wt % to 50 wt %, based in each case on the weight of said preparation.

Organic solvents or a mixture of solvents having a boiling point below 400° C. can be contained as additional co-solvents, in a quantity from 0.1 to 15 weight percent, preferably from 1 to 10 weight percent, based in each case on the weight of said preparation. Unbranched or branched hydrocarbons such as pentane, hexane, isopentane, and cyclic hydrocarbons such as cyclopentane and cyclohexane, are particularly suitable as additional co-solvents. Further particularly preferred water-soluble solvents are polyethylene glycol and propylene glycol, in a quantity of up to 30 wt % based on the weight of said preparation.

The addition in particular of propylene glycol and/or polyethylene glycol and/or polypropylene glycol increases the flexibility of the hold formed when the aerosol composition according to the present invention is used. If a flexible hold is desired, the agents according to the present invention therefore contain by preference 0.01 to 30 wt % polyethylene glycol and/or polypropylene glycol, based on the weight of said preparation.

The agents preferably have a pH from 2 to 11. Particularly preferably, the pH range is between 2 and 8. The indications as to pH refer, for purposes of this document, to the pH at 25° C. unless otherwise noted.

It has been possible to increase the effects according to the present invention by adding at least one (C₂ to C₆) trialkyl citrate to the preparation according to the present invention. It is therefore preferred according to the present invention if the preparation additionally contains at least one compound of formula (E)

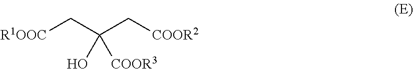

in which

R¹, R², and R³, mutually independently, denote a (C₂ to C₆) alkyl group.

Examples of a (C₂ to C₆) alkyl group according to formula (E) are methyl, ethyl, isopropyl, n-propyl, n-butyl, sec-butyl, isobutyl, tert-butyl, n-pentyl, neopentyl, isopentyl, n-hexyl.

Triethyl citrate has proven to be a particularly preferred compound of formula (E).

The preparation according to the present invention contains the compounds of formula (E) preferably in a quantity from 0.01 to 1 wt %, in particular from 0.05 to 0.3 wt %, based in each case on the weight of said preparation.

The preparations according to the present invention preferably contain, by preference additionally, at least one surfactant; nonionic, anionic, cationic, and ampholytic surfactants are suitable in principle. The group of the ampholytic or also amphoteric surfactants encompasses zwitterionic surfactants and ampholytes. The surfactants can, according to the present invention, already have an emulsifying effect. The use of at least one nonionic surfactant and/or of at least one cationic surfactant is preferred in the context of this embodiment of the invention.

The additional surfactants are contained in the preparation according to the present invention preferably in a quantity from 0.01 wt % to 5 wt %, particularly preferably from 0.05 wt % to 0.5 wt %, based in each case on the weight of said preparation.

It has proven to be particularly preferred if the preparations according to the present invention additionally contain at least one nonionic surfactant.

Nonionic surfactants contain as a hydrophilic group, for example, a polyol group, a polyalkylene glycol ether group, or a combination of a polyol and polyglycol ether group. Such compounds are, for example:
addition products of 2 to 100 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group,
addition products, end-capped with a methyl or C₂ to C₆ alkyl residue, of 2 to 50 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group, such as, for example, the grades obtainable under the marketing designations Dehydol® LS, Dehydol® LT (Cognis),
C₁₂ to C₃₀ fatty acid mono- and diesters of addition products of 1 to 30 mol ethylene oxide with glycerol,
addition products of 5 to 60 mol ethylene oxide with castor oil and hardened castor oil,
polyol fatty acid esters such as, for example, the commercial product Hydagen® HSP (Cognis), or Sovermol® grades (Cognis),
alkoxylated triglycerides,
alkoxylated fatty acid alkyl esters of formula (T-I)

R¹CO—(OCH₂CHR²)_{w}OR³ (T-I),

in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms, and w denotes numbers from 1 to 20,
amine oxides,
hydroxy mixed ethers such as those described, for example, in German Application 19738866,
sorbitan fatty acid esters and addition products of ethylene oxide with sorbitan fatty acid esters, for example the polysorbates,
sugar fatty acid esters and addition products of ethylene oxide with sugar fatty acid esters,
addition products of ethylene oxide with fatty acid alkanolamides and fatty amines,
sugar surfactants of the alkyl and alkenyl oligoglycoside types, in accordance with formula (T-II)

R⁴O-[G]ₚ (T-II),

in which R⁴ denotes an alkyl or alkenyl residue having 4 to 22 carbon atoms, G denotes a sugar residue having 5 or 6 carbon atoms, and p denotes numbers from 1 to 10. They can be obtained in accordance with relevant methods of preparative organic chemistry.

The alkyl and alkenyl oligoglycosides can be derived from aldoses resp. ketoses having 5 or 6 carbon atoms, preferably from glucose. The preferred alkyl and/or alkenyl oligoglycosides are thus alkyl and/or alkenyl oligoglucosides. The index number p in the general formula (T-II) indicates the degree of oligomerization (DP), i.e. the distribution of mono- and oligoglycosides, and denotes a number between 1 and 10. Whereas p in the individual molecule must always be a whole number, and here can assume especially the values p=1 to 6, the value p for a specific alkyl oligoglycoside is an analytically ascertained calculated value that usually represents a fractional number. Alkyl and/or alkenyl oligoglycosides having an average degree of oligomerization p from 1.1 to 3.0 are preferably used. In terms of applications engineering, those alkyl and/or alkenyl oligoglycosides whose degree of oligomerization is less than 1.7, and in particular between 1.2 and 1.4, are preferred. The alkyl resp. alkenyl residue R⁴ can be derived from primary alcohols having 4 to 11, preferably 8 to 10 carbon atoms. Typical examples are butanol, hexanol, octanol, decanol, and undecyl alcohol as well as industrial mixtures thereof, such as those obtained, for example, upon hydrogenation of industrial fatty acid methyl esters or in the course of the hydrogenation of aldehydes from Roelen oxosynthesis. Preferred are alkyl oligoglucosides of chain length C₈ to C₁₀ (DP=1 to 3), which occur as the first runnings upon distillational separation of industrial C₈ to C₁₈ coconut fatty alcohol and can be contaminated with a proportion of less than 6 wt % C₁₂ alcohol, as well as alkyl oligoglucosides based on industrial C_{9/11} oxoalcohols (DP=1 to 3). The alkyl resp. alkenyl residue R¹⁵ can furthermore also be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and industrial mixtures thereof, which can be obtained as described above. Alkyl oligoglucosides based on hardened C_{12/14} coconut alcohol having a DP of 1 to 3 are preferred.

The alkylene oxide addition products with saturated linear fatty alcohols and fatty acids having respectively 2 to 100 mol ethylene oxide per mol fatty alcohol resp. fatty acid have proven to be particularly preferred nonionic surfactants. Preparations having outstanding properties are likewise obtained when they contain, as nonionic surfactants, C₁₂ to C₃₀ fatty acid mono- and diesters of addition products of 1 to 30 mol ethylene oxide with glycerol and/or addition products of 5 to 60 mol ethylene oxide with castor oil and hardened castor oil.

In the case of the surfactants that represent addition products of ethylene oxide and/or propylene oxide with fatty alcohols, or derivatives of said addition products, both products having a “normal” homolog distribution and those having a restricted homolog distribution can be used. A “normal” homolog distribution is understood as mixtures of homologs that are obtained upon reaction of fatty alcohol and alkylene oxide using alkali metals, alkali metal hydroxides, or alkali metal alcoholates as catalysts. Restricted homolog distributions, on the other hand, are obtained when, for example, hydrotalcites, alkaline-earth metal salts of ethercarboxylic acids, or alkaline-earth metal oxides, hydroxides, or alcoholates are used as catalysts. The use of products having a restricted homolog distribution can be preferred.

Very particularly preferably, the preparations according to the present invention contain as a surfactant at least one addition product of 15 to 100 mol ethylene oxide, in particular 15 to 50 mol ethylene oxide, with a linear or branched (in particular linear) fatty alcohol having 8 to 22 carbon atoms. This refers very particularly preferably to Ceteareth-15, Ceteareth-25, or Ceteareth-50, which are marketed as Eumulgin® CS 15 (COGNIS), Cremophor A25 (BASF SE), resp. Eumulgin® CS 50 (COGNIS).

All anionic surface-active substances suitable for use on the human body are, in principle, appropriate as anionic surfactants. These are characterized by an anionic group imparting water solubility, for example a carboxylate, sulfate, sulfonate, or phosphate group, and a lipophilic alkyl group having approximately 8 to 30 carbon atoms. Glycol ether or polyglycol ether groups, ester, ether, and amide groups, and hydroxyl groups can additionally be contained in the molecule. Examples of suitable anionic surfactants are, in each case in the form of the sodium, potassium, and ammonium and the mono-, di, and trialkanolammonium salts having 2 to 4 carbon atoms in the alkanol group:
linear and branched fatty acids having 8 to 30 carbon atoms (soaps);
ethercarboxylic acids of the formula R—O—(CH₂—CH₂O)ₓ—CH₂—COOH, in which R is a linear alkyl group having 8 to 30 carbon atoms and x=0 or is 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid mono- and dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
linear alkanesulfonates having 8 to 24 carbon atoms;
linear alpha-olefinsulfonates having 8 to 24 carbon atoms;
alpha-sulfo fatty acid methyl esters of fatty acids having 8 to 30 carbon atoms;
alkyl sulfates and alkyl polyglycol ether sulfates of the formula R—O—(CH₂—CH₂—O)ₓ—OSO₃H, in which R is a preferably linear alkyl group having 8 to 30 carbon atoms and x=0 or is 1 to 12;
mixtures of surface-active hydroxysulfonates;
sulfated hydroxyalkylpolyethylene and/or hydroxyalkylenepropylene glycol ethers;
sulfonates of unsaturated fatty acids having 8 to 24 carbon atoms and 1 to 6 double bonds;
esters of tartaric acid and citric acid with alcohols representing addition products of approximately 2 to 15 molecules of ethylene oxide and/or propylene oxide with fatty alcohols having 8 to 22 carbon atoms;
alkyl and/or alkenyl ether phosphates of formula (T-V)

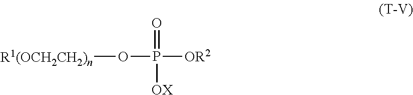

in which R¹ preferably denotes an aliphatic hydrocarbon residue having 8 to 30 carbon atoms, R² denotes hydrogen, a (CH₂CH₂O)ₙR¹ residue, or X, n denotes numbers from 1 to 10, and X denotes hydrogen, an alkali or alkaline-earth metal, or NR³R⁴R⁵R⁶ where R³ to R⁶, mutually independently, denote hydrogen or a C₁ to C₄ hydrocarbon residue;
sulfated fatty acid alkylene glycol esters of formula (T-VI)

R⁷CO(AlkO)ₙSO₃M (T-VI)

in which R⁷CO denotes a linear or branched, aliphatic, saturated and/or unsaturated acyl residue having 6 to 22 carbon atoms, Alk denotes CH₂CH₂, CHCH₃CH₂, and/or CH₂CHCH₃, n denotes numbers from 0.5 to 5, and M denotes a cation;
monoglyceride sulfates and monoglyceride ether sulfates of formula (T-VII)

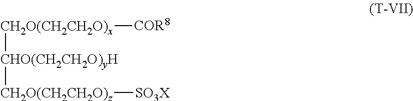

in which R⁸CO denotes a linear or branched acyl residue having 6 to 22 carbon atoms, x, y, and z in total denote 0 or numbers from 1 to 30, preferably 2 to 10, and X denotes an alkali or alkaline-earth metal. Typical examples of monoglyceride (ether) sulfates suitable for purposes of the invention are the reaction products of lauric acid monoglyceride, coconut fatty acid monoglyceride, palmitic acid monoglyceride, stearic acid monoglyceride, oleic acid monoglyceride, and tallow fatty acid monoglyceride, as well as ethylene oxide adducts thereof with sulfur trioxide or chlorosulfonic acid in the form of their sodium salts. It is preferable to use monoglyceride sulfates of formula (T-VII) in which R⁸CO denotes a linear acyl residue having 8 to 18 carbon atoms,
amide ethercarboxylic acids;
condensation products of C₈ to C₃₀ fatty alcohols with protein hydrolysates and/or amino acids and derivatives thereof, known to one skilled in the art as protein fatty acid condensates, such as, for example, the Lamepon® grades, Gluadin® grades, Hostapon® KCG, or the Amisoft® grades.

Cationic surfactants of the quaternary ammonium compound, esterquat, and amidoamine types are also usable according to the present invention. Preferred quaternary ammonium compounds are ammonium halides, in particular chlorides and bromides, such as alkyltrimethylammonium chlorides, dialkyldimethylammonium chlorides, and trialkylmethylammonium chlorides. The long alkyl chains of these surfactants preferably have 10 to 18 carbon atoms, for example as in cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Further preferred anionic surfactants are the imidazolium compounds known by the INCI names Quaternium-27 and Quaternium-83.

“Zwitterionic surfactants” refers to those surface-active compounds that carry in the molecule at least one quaternary ammonium group and at least one —COO⁽⁻⁾ or SO₃⁽⁻⁾ group. Particularly suitable zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines, having in each case 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Ampholytes” are understood to be those surface-active compounds that contain in the molecule, besides a C₈ to C₂₄ alkyl or acyl group, at least one free amino group and at least one —COOH or —SO₃H group, and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Particularly preferred ampholytes are N-cocalkylaminopropionate, cocacylaminoethylaminopropionate, and C₁₂ to C₁₈ acyl sarcosine.

In the context of an embodiment preferred according to the present invention, the preparations additionally contain at least one silicone oil. Included among the silicone oils are, for example, dialkyl- and alkylarylsiloxanes, such as e.g. cyclopentasiloxane, cyclohexasiloxane, dimethylpolysiloxane, and methylphenylpolysiloxane, but also hexamethyldisiloxane, octamethyltrisiloxane, and decamethyltetrasiloxane. Volatile linear silicone oils are particularly preferred, in particular hexamethyldisiloxane (L₂), octamethyltrisiloxane (L₃), decamethyltetrasiloxane (L₄), dodecamethylpentasiloxane (L₅), as well as any two-, three-, and four-member mixtures of L₂, L₃, L₄ and/or L₅ such as those contained, for example, in the commercial products DC 2-1184 Fluid, Dow Corning® 200 (0.65 cSt), and Dow Corning® 200 (1.5 cSt) of Dow Corning, the kinematic viscosity values referring to a temperature of 25° C.

Besides the aforementioned substances, usually referred to as “volatile” silicone oils, and besides the aforementioned volatile non-silicone oils, preparations particularly preferred according to the present invention can furthermore contain at least one nonvolatile cosmetic oil selected from nonvolatile silicone oils and nonvolatile non-silicone oils.

Preferred nonvolatile silicone oils are selected from higher-molecular-weight dimethylpolysiloxanes, obtainable commercially e.g. under the designation Dow Corning® 190, Dow Corning® 200 Fluid, having kinematic viscosities (25° C.) in the range from 5 to 100 cSt, preferably 5 to 50 cSt, or even 5 to 10 cSt, and Baysilon® 350 M (having a kinematic viscosity (25° C.) of approximately 350 cSt.

Further nonvolatile non-silicone oils particularly preferred according to the present invention are selected from the esters of linear or branched, saturated or unsaturated fatty alcohols having 2 to 30 carbon atoms with linear or branched, saturated or unsaturated fatty acids having 2 to 30 carbon atoms, which can be hydroxylated. These include hexyldecyl stearate (e.g. Eutanol® G 16 S), hexyldecyl laurate, isodecyl neopentanoate, isononyl isononanoate, 2-ethylhexyl palmitate and 2-ethylhexyl stearate, in particular 2-ethylhexyl palmitate. Also preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

The Examples that follow are intended to illustrate the subject matter of the present Application without limiting it thereto.

### EXAMPLES

1.0 Hair Sprays

The quantity indications are percentages by weight based on the total weight of the agent (i.e. of the aerosol composition according to the present invention).

| | A | B | C | D |
|---|---|---|---|---|
| Amphomer | 4.00 | — | — | — |
| Luviskol ® VA37 | — | 8.00 | — | — |
| Aquaflex SF 40 | — | — | 10.00 | — |
| Advantage LC-E | — | — | — | 10.81 |
| 2-amino-2-methylpropanol | 0.71 | — | — | — |
| Water (dist.) | 1.00 | 1.00 | 1.00 | 1.00 |
| Ethanol | to 100 | to 100 | to 100 | to 100 |
| E-1,3,3,3-tetrafluoropent-1-ene | 50.00 | 50.00 | 50.00 | 50.00 |

| | E | F | G | H | I | J |
|---|---|---|---|---|---|---|
| Ultrahold Strong | 4.00 | — | — | — | — | — |
| Ultrahold 8 | — | 4.00 | — | — | — | — |
| Acudyne LT 120 | — | — | 8.50 | — | — | — |
| Resyn 28-2930 | — | — | — | 4.00 | — | — |
| Luvimer 100 P | — | — | — | — | 4.00 | — |
| Luviset Shape | — | — | — | — | — | 10.81 |
| 2-amino-2- | 0.50 | 0.40 | 0.68 | 0.37 | 0.94 | — |
| methylpropanol | | | | | | |
| Water (dist.) | 1.00 | 1.00 | 1.00 | 1.00 | 1.00 | 1.00 |
| Ethanol | to 100 | to 100 | to 100 | to 100 | to 100 | to 100 |
| E-1,3,3,3-tetra- | 50.00 | 50.00 | 50.00 | 50.00 | 50.00 | 50.00 |
| fluoropent-1-ene | | | | | | |

| Raw material | K | L | M |
|---|---|---|---|
| Polydimethylsiloxane | 3.00 | 2.00 | 10.00 |
| Trisiloxane | to 100 | — | to 100 |
| Isopropyl alcohol | — | to 100 | — |
| E-1,3,3,3-tetrafluoropent-1-ene | 55.00 | 90.00 | 65.00 |

2.0 Hair Foams

The quantity indications are percentages by weight based on the total weight of the preparation of the aerosol composition according to the present invention.

| Raw materials | A | B | C | D | E | F | G | H |
|---|---|---|---|---|---|---|---|---|
| Aquastyle ® 300 | 6.0 | 6.0 | 2.0 | 5.0 | 5.0 | 4.0 | 4.0 | 2.0 |
| Celquat ® L 200 | 1.0 | 2.0 | 1.0 | — | — | 1.0 | 1.0 | — |
| Styleze ® W-10 | — | — | — | 4.0 | 4.0 | — | — | 3.5 |
| Styleze ® CC 10 | — | — | — | — | — | 6.0 | 2.0 | 2.0 |
| Luviskol ® VA 64 W | 8.0 | — | — | 6.0 | — | — | — | — |
| Luviskol ® K 85 | — | 6.0 | — | — | 8.0 | — | — | — |
| Luviset ® Clear | — | — | 5.0 | — | — | — | — | — |
| Acudyne ® SCP | — | — | — | — | — | — | 3.0 | — |
| PEG-40 Hydrogenated Castor | 0.2 | 0.2 | 0.1 | 0.1 | 0.2 | 0.2 | 0.15 | 0.2 |
| Oil | | | | | | | | |

| | |
|---|---|
| Water | 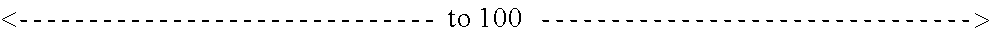 |

| Raw materials | J | K | L | M | N | O | P | Q |
|---|---|---|---|---|---|---|---|---|
| Aquastyle ® 300 | 3.0 | 3.0 | 3.0 | 6.0 | 2.0 | 3.0 | 3.0 | 3.0 |
| Luviquat ® Supreme | 5.0 | 4.0 | — | — | — | — | — | — |
| Styleze ® W-10 | — | — | — | — | — | — | 4.0 | 4.0 |
| Gafquat ® 755 N PW | — | — | 5.0 | 2.0 | 5.0 | 5.0 | — | — |
| Luviskol ® VA 64 W | 6.0 | — | — | 5.0 | — | — | — | — |
| Luviskol ® K 85 | — | 6.0 | 5.0 | — | — | — | — | — |
| Luviset ® Clear | — | — | — | — | 6.0 | — | — | — |
| Styleze ® CC 10 | — | — | — | — | — | 3.0 | 3.0 | 3.0 |
| PEG-40 Hydrogenated Castor | 0.2 | 0.2 | 0.1 | 0.1 | 0.2 | 0.2 | 0.1 | 0.2 |
| Oil | | | | | | | | |

| | |
|---|---|
| Water | 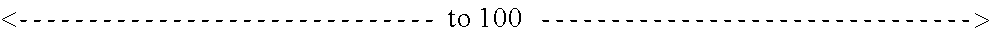 |

Formulations A to Q were each introduced into an aerosol vessel that meets the following technical parameters: aluminum reservoir vessel with product no. 522983 PV10697 valve of the Precision company (Deutsche Prazisions-Ventil GmbH).

The aerosol vessel was filled with E-1,3,3,3-tetrafluoroprop-1-ene as propellant, so as to yield a weight ratio of formulation to propellant gas of 92 to 8.

All formulations produced an outstanding flexible hairstyle hold after application to the hair. The hair obtained very good care. When applied as an aerosol foam, a voluminous foam was obtained that did not collapse significantly until applied on the hair.

3.0 Hair Spray Wax

| | A |
|---|---|
| Cetyl palmitate | 0.80 |
| Beeswax | 0.15 |
| Petrolatum | 16.0 |
| Trilaureth-4 phosphate | 1.80 |
| Microcrystalline paraffin wax | 0.20 |
| Sunflower oil | 0.2 |
| Water (dist.) | 0.20 |
| Ethanol | to 100 |
| E-1,3,3,3-tetrafluoropent-1-ene | 50.00 |

List of Raw Materials Used:

| | |
|---|---|
| Advantage ® LC-E | Vinylcaprolactam/vinylpyrrolidone/diimethylaminoethyl methacrylate |
| | copolymer (approx. 35-39% solids in Ethanol; INCI name: Vinyl |
| | Caprolactam/VP/Dimethylaminoethyl Methacrylate Copolymer, |
| | Alcohol, Lauryl Pyrrolidone) (ISP) |
| Aquastyle ® 300 | Copolymer of N-vinylpyrrolidone/N-vinylcaprolactam/N- |
| | (3-dimethylaminopropyl) methacrylamide, and 3-(methacryloylamino) |
| | propyllauryldimethylammonium chloride (active substance 30 wt % |
| | in water/ethanol, INCI name: Polyquaternium-69) (ISP) |
| Acudyne ® SCP | Copolymer of acrylamide and 2-acrylamido-2-methyl-1-propanesulfonate |
| | sodium salt (approx. 25 to 27 wt % active substance in water, |
| | INCI name: Acrylamide/Sodium Acryloyldimethyltaurate/Acrylic Acid |
| | Copolymer) (Rohm & Haas) |
| Acudyne ® LT 120 | Copolymer of succinic acid C₁ to C₂ alkyl esters, hydroxyalkyl acrylate, |
| | and at least one monomer from among acrylic acid, methacrylic acid, |
| | and simple esters thereof (approx. 46-47.5% solids in water; INCI name: |
| | Acrylates/C1-2 Succinates/Hydroxyacrylates Copolymer) (ISP) |
| Amphomer ® | INCI name: Octylacrylamide/Acrylates/Butyl-aminoethyl Methacrylate |
| | Copolymer (National Starch) |
| Celquat ® L 200 | Quaternized cellulose derivative (INCI name: Polyquaternium-4) |
| | (National Starch) |
| Gafquat ® 755 N PW | Dimethylaminoethyl methacrylate/vinylpyrrolidone copolymer, |
| | quaternized with diethyl sulfate (approx. 19% solids in water; |
| | INCI name: Polyquaternium-11) (ISP) |
| Luviset ® Clear | Vinylpyrrolidone/methacrylamide/vinylimidazole copolymerizate |
| | (19-21% solids in water; INCI name: VP/Methacrylamide/Vinyl |
| | Imidazole Copolymer) (BASF) |
| Luviskol ® K85 | Polyvinylpyrrolidone (approx. 20% solids in water; INCI name: |
| | PVP) (BASF) |
| Luviskol ® VA 64 W | Copolymer of vinylpyrrolidone and vinyl acetate (48-52% active |
| | substance in water, INCI name: VP/VA Copolymer) (BASF) |
| Luviskol ® VA37 | Vinylpyrrolidone/vinyl acetate copolymer (30:70) (approx. 48-52% |
| | solids in isopropanol; INCI name: VP/VA Copolymer, Isopropyl |
| | Alcohol) (BASF) |
| Luviquat ® FC 370 | 3-Methyl-1-vinylimidazolium chloride/vinylpyrrolidone |
| | copolymerizate (30:70) (38-42% solids in water; INCI name: |
| | Polyquaternium-16) (BASF) |
| Luviquat ® Supreme | Vinylpyrrolidone/methacrylamide/vinylimidazole/vinylimidazolium |
| | methosulfate copolymerisate (55:29:10:6) (19-21% solids in water; |
| | INCI name: Polyquaternium-68) (BASF) |
| Luvimer 100 P | Terpolymer of butyl acrylate, ethyl acrylate, and methacrylic acid |
| | (INCI name: Acrylates Copolymer, K-value: 34-40) (BASF SE) |
| Luviset Shape | INCI name: Polyacrylate-22 (active substance: 32-36 wt %) (BASF SE) |
| Resyn 28-2930 | Copolymer of vinyl acetate, crotonic acid, and vinyl neodecanoate |
| | (INCI name: VA/Crotonates/Vinyl Neodecanoate Copolymer) |
| | (Akzo Nobel) |
| Styleze ® W-10 | Copolymer of N-vinylpyrrolidone, N,N-dimethylaminopropyl |
| | methacrylamide, and N,N-dimethyl-N-dodecylammoniopropyl |
| | methacrylamide chloride (approx. 9 to 11% active substance, |
| | INCI name: Polyquaternium-55) (ISP) |
| Styleze ® CC 10 | Copolymer of N-vinylpyrrolidone and N,N-dimethylaminopropyl |
| | methacrylamide (approx. 9 to 11% active substance, INCI name: |
| | VP/DMAPA Acrylates Copolymer) (ISP) |
| Ultrahold 8 | Copolymer of acrylic acid, ethyl acrylate, and N-tert-butylamide |
| | (INCI name: Acrylates/t-Butylacrylamide Copolymer, K-Value |
| | 22 to 32) (BASF SE) |
| Ultrahold Strong | Copolymer of acrylic acid, ethyl acrylate, and N-tert-butylamide |
| | (INCI name: Acrylates/t-Butylacrylamide Copolymer, K-Value |
| | 35-45) (BASF SE). |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An aerosol composition encompassing
a preparation containing, in a cosmetically acceptable carrier, at least one setting active substance, and
at least one propellant selected from at least cone compound having 3 to 10 carbon atoms in accordance with formula (I) 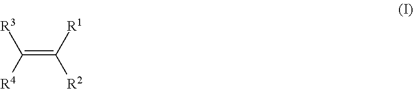
in which the residues R¹, R², R³, and R⁴ signify, mutually independently, a hydrogen atom, a bromine atom, a fluorine atom, or a (C₁ to C₆) alkyl group substituted with at least one fluorine atom,
or two of the residues R¹, R², R³, and R⁴ form a five- or six-membered ring, with the provision that:
at least one of the residues R¹, R², R³, or R⁴ denotes a hydrogen atom or a fluorine atom, and
at least one of the residues R¹, R², R³, or R⁴ denotes a (C₁ to C₆) alkyl group substituted with at least one fluorine atom, or at least two of the residues R¹, R², R³, and R⁴ form a five- or six-membered ring.

2. The aerosol composition according to claim 1, wherein R¹, R², R³, or R⁴ in accordance with formula (I) signify, mutually independently, a hydrogen atom, a fluorine atom, or a (C₁ to C₆) alkyl group substituted with at least one fluorine atom,
with the provision that:
at least one of the residues R¹, R², R³, or R⁴ denotes a hydrogen atom or a fluorine atom, and
at least one of the residues R¹, R², R³, or R⁴ denotes a (C₁ to C₆) alkyl group substituted with at least one fluorine atom.

3. The aerosol composition according to claim 1, wherein the propellant of formula (I) is selected from at least one compound of the group consisting of
compounds of the formula E-R¹CH═CHR² or Z-R¹CH═CHR², in which R¹ and R² represent, mutually independently, a perfluorinated C₁ to C₆ alkyl group,
CF₃CF═CHF, CF₃CH═CF₂, CHF₂CF═CF₂, CHF₂CH═CHF, CF₃CF═CH₂, CF₃CH═CHF, CH₂FCF═CF₂, CHF₂CH═CF₂, CHF₂CF═CHF, CHF₂CF═CH₂, CF₃CH═CH₂, CH₃CF═CF₂, CH₂FCH═CF₂, CH₂FCF═CHF, CHF₂CH═CHF, CF₃CF═CFCF₃, CF₃CF₂CF═CF₂, CF₃CF═CHCF₃, CF₃CF₂CF═CH₂, CF₃CH═CHF₃, CF₃CF₂CH═CH₂, CF₂═CHCF₂CF₃, CF₂═CHCF₂CF₃, CF₂═CFCHFCF₃, CF₂═CFCF₂CHF₂, CHF₂CH═CHCF₃, (CF₃)₂C═CHCF₃, CF₃ CF═CHCF₂CF₃, CF₃CH═CFCF₂CF₃, (CF₃)₂CFCH═CH₂, CF₃CF₂CF₂CH═CH₂, CF₃(CF₂)₃CF═CF₂, CF₃CF₂CF═CFCF₂CF₃, (CF₃)₂C═C(CF₃)₂, (CF₃)₂CFCF═CHCF₃, CF₂═CFCF₂CH₂F, CF₂═CFCHFCHF₂, CH₂═C(CF₃)₂, CH₂CF₂CF═CF₂, CH₂FCF═CFCHF₂, CH₂FCF₂CF═CF₂, CF₂═C(CF₃)(CH₃), CH₂═C(CHF₂)(CF₃), CH₂═CHCF₂CHF₂, CF₂═C(CHF₂)(CH₃), CHF═C(CF₃)(CH₃), CH₂═C(CHF₂)₂, CF₃CF═CFCH₃, CH₃CF═CHCF₃, CF₂═CF(CF₂)₂CF₃, CHF═CF(CF₂)₂CF₃, CF₂═CH(CF₂)₂CF₃, CF₂═CF(CF₂)₂CHF₂, CHF₂CF═CFCF₂CF₃, CF₃CF═CFCF₂CHF₂, CF₃CF═CFCHFCF₃, CHF═CFCF(CF₃)₂, CF₂═CFCH(CF₃)₂, CF₃CH═C(CF₃)₂, CF₂═CHCF(CF₃)₂, CH₂═CF(CF₂)₂CF₃, CHF═CF(CF₂)₂CHF₂, CH₂═C(CF₃)C₂F₅, CF₂═CHCH(CF₃)₂, CHF═CHCF(CF₃)₂, CF₂═C(CF₃)CH₂CF₃, CH₂═CF(CF₂)₂CHF₂, CF₂═CHCF₂CH₂CF₃, CF₃CF═C(CF₃)CH₃, CH₂═CFCH(CF₃)₂, CHF═CHCH(CF₃)₂, CH₂FCH═C(CF₃)₂, CH₃CF═C(CF₃)₂, CH₂═CHCF₂CHFCF₃, CH₂═C(CF₃)CH₂CF₃, (CF₃)₂C═CHC₂F₅, CH₂═CHC(CF₃)₃, (CF₃)₂C═C(CH₃)CF₃, CH₂═CFCF₂CH(CF₃)₂, CF₃CF═C(CH₃)C₂F₅, CF₃CH═CHCH(CF₃)₂, CH₂═CH(CF₂)₃CHF₂, (CF₃)₂C═CHCF₂CH₃, CH₂═C(CF₃)CH₂C₂F₅, CH₂═CHCH₂CF₂CF₂CF₃, C₂F₅CF═CFC₂H₅, CH₂═CHCH₂CF(CF₃)₂, CF₃CF═CHCH(CF₃)(CH₃), (CF₃)₂C═CFC₂H₅, cyclo-CF₂CF₂CF₂CH═CH—, cyclo-CF₂CF₂CH═CH—, CF₃CF₂CF₂C(CH₃)═CH₂, CF₃CF₂CF₂CH═CHCH₃, cyclo-CF₂CF₂CF═CF—, cyclo-CF₂CF═CFCF₂CF₂—, cyclo-CF₂CF═CFCF₂CF₂CF₂—, CF₃CF₂CF₂CF₂CH═CH₂, CF₃CH═CHC₂F₅, C₂F₅CH═CHC₂F₅, CF₃CH═CHCF₂CF₂CF₃, CF₃CF═CFC₂F₅, CF₃CF═CFCF₂CF₂CF₂CF₃, C₂F₅CF═CFCF₂CF₂CF₃, CF₃CH═CFCF₂CF₂CF₂CF₃, CF₃CF═CHCF₂CF₂CF₂CF₃, C₂F₅CH═CFCH₂CH₂CH₃, C₂F₅CF═CHCF₂CF₂CF₃, CF₃CF₂CF₂CF═CHCH₃, C₂F₅CF═CHCH₃, (CF₃)₂C═CHCH₃, CF₃C(CH₃)═CHCF₃, CHF═CFC₂F₅, CHF₂CF═CFCF₃, (CF₃)₂C═CHF, CH₂FCF═CFCF₃, CHF═CHC₂F₅, CHF₂CH═CFCF₃, CHF═CFCHFCF₃, CF₃CH═CFCHF₂, CHF═CFCF₂CHF₂, CHF₂CF═CFCHF₂, CH₂CF═CFCF₃, CH₂FCH═CFCF₃, CH₂═CFCHFCF₃, CH₂═CFCF₂CHF₂, CF₃CH═CFCH₂F, CHF═CFCH₂CF₃, CHF═CHCHFCF₃, CHF═CHCF₂CHF₂, CHF₂CF═CHCHF₂, CHF═CFCHFCHF₂, CF₃CF═CHCH₃, CF₂═CHCF₂Br, CHF═CBrCHF₂, CHBr═CHCF₃, CF₃CBr═CFCF₃, CH₂═CBrC₂F₅, CHBr═CHC₂F₅, CH₂═CH(CF₂)₂Br, CH₂═CHCBrFCF₃, CH₃CBr═CHCF₃, CF₃CBr═CHCH₃, (CF₃)₂C═CHBr, CF₃CF═CBrC₂F₅, E-CHF₂CBr═CFC₂F₅, Z—CHF₂CBr═CFC₂F₅, CF₂═CBrCHFC₂F₅, (CF₃)₂CFCBr═CH₂, CHBr═CF(CF₂)₂CHF₂, CH₂═CBrCF₂CF₂CF₃, CF₂═C(CH₂Br)CF₃, CH₂═C(CBrF₂)CF₃, (CF₃)₂CHCH═CHBr, (CF₃)₂C═CHCH₂Br, CH₂═CHCF(CF₃)CBrF₂, CF₂═CHCF₂CH₂CBrF₂, CFBr═CHCF₃, CFBr═CFCF₃, and CH₂═CBrCF₂CF₂CF₂CF₃, in each case in the E form or the Z form.

4. The aerosol composition according to claim 1, wherein the aerosol composition includes E-CF₃CH═CHF (E-1,3,3,3-tetrafluoroprop-1-ene) as a propellant of formula (I).

5. The aerosol composition according to claim 1, wherein the propellant of formula (I) is included in a quantity from 1 to 60 wt %.

6. The aerosol composition according to claim 1, wherein the preparation includes as a setting active substance at least one nonionic setting polymer and at least one cationic setting polymer.

7. The aerosol composition according to claim 1, wherein the preparation includes at least one wax as a setting active substance.

8. The aerosol composition according to claim 1, wherein the preparation additionally comprises at least one surfactant.

9. The aerosol composition according to claim 1, wherein the preparation is packaged in an aerosol dispensing container.
